(19) United States
(12) Patent Application Publication
(10) Pub. No.: US 2025/0040831 A1
(43) Pub. Date: Feb. 6, 2025

(54) MOBILITY ESTIMATION DEVICE, MOBILITY ESTIMATION SYSTEM, MOBILITY ESTIMATION METHOD, AND RECORDING MEDIUM

(71) Applicant: NEC Corporation, Minato-ku, Tokyo (JP)

(72) Inventors: Chenhui HUANG, Tokyo (JP); Fumiyuki NIHEY, Tokyo (JP); Zhenwei WANG, Tokyo (JP); Hiroshi KAJITANI, Tokyo (JP); Yoshitaka NOZAKI, Tokyo (JP); Kenichiro FUKUSHI, Tokyo (JP)

(73) Assignee: NEC Corporation, Minato-ku, Tokyo (JP)

(21) Appl. No.: 18/716,606

(22) PCT Filed: Dec. 27, 2021
(86) PCT No.: PCT/JP2021/048552

§ 371 Date: Jun. 5, 2024

(51) Int. Cl. A61B 5/11

(52) U.S. Cl. CPC A61B 5/11

(57) ABSTRACT

Provided is a mobility estimation device that includes a data acquisition unit that acquires feature amount data including a feature amount used for estimating a mobility of a user, the feature amount data being extracted from sensor data regarding a movement of a foot of the user, a storage unit that stores an estimation model that outputs a mobility index based on an input of the feature amount data, an estimation unit that inputs the acquired feature amount data to the estimation model and estimates the mobility of the user in accordance with the mobility index output from the estimation model, and an output unit that outputs information regarding the estimated mobility of the user.

### TECHNICAL FIELD

The present disclosure relates to a mobility estimation device and the like that estimate a mobility using sensor data regarding a motion of a foot.

### BACKGROUND ART

With increasing interest in healthcare, services for providing information in accordance with features (also referred to as gait) included in a gait pattern have attracted attention. For example, a technique for analyzing a gait based on sensor data measured by a sensor mounted on footwear such as shoes has been developed. In time-series data of the sensor data, features of a gait event (also referred to as a walking event) related to a physical condition appear.

PTL 1 discloses a device that detects an abnormality of a foot based on features of a gait of a pedestrian. The device of PTL 1 extracts a characteristic gait feature amount in a gait of a pedestrian wearing footwear by using data acquired from a sensor installed on the footwear. The device of PTL 1 detects an abnormality of a pedestrian walking while wearing the footwear based on the extracted gait feature amount. For example, the device of PTL 1 extracts a feature portion regarding hallux valgus from gait waveform data for one gait cycle. The device of PTL 1 estimates the progress state of hallux valgus using the extracted gait feature amount of the feature portion.

PTL 2 discloses a gait analysis device that estimates mobility according to acceleration measured by an accelerometer installed on a waist portion. The device of PTL 2 measures a temporal change in acceleration of at least one of an up-down direction, a front-back direction, or a left-right direction of the waist during a gait. The device of PTL 2 extracts a specific period in which a specific gait motion is performed during a gait based on a temporal change in any acceleration. The device of PTL 2 calculates an estimated index related to mobility during a gait based on a temporal change in any acceleration in a specific period. The device of PTL 2 estimates the mobility using the relationship between the calculated estimated index and the estimated index prepared in advance, and the mobility.

Actions such as walking, going up and down stairs, changing directions, straddling, and standing and sitting are important actions in daily life. The ability to walk, go up and down stairs, change directions, straddle, stand and sit, and the like is called mobility. Mobility is deeply related to Quality of Life (QoL). As a test for evaluating mobility, there is a time up and go (TUG) test. The TUG test includes three parts: standing, sitting, walking, and changing direction. The subject stands up from a seated state on a chair, walks toward a mark 3 m (meter) ahead, changes direction at the position of the mark, walks toward the seated chair, and sits on the chair. The performance of the TUG test is evaluated by the time taken for this series of operations.

NPL 1 reports the results of verification of the TUG test for healthy young people around 20 years old and healthy elderly people around 70 years old. In NPL 1, the ratio of each of standing and sitting, walking, and changing direction constituting the TUG test is verified. In the verification of NPL 1, for the healthy elderly, the ratio of standing and sitting was 18% (percent), the ratio of direction change was 12%, and the ratio of reciprocating gait was 70%.

NPL 2 reports a case where a muscle activity of a support-side lower limb at the time of the direction changing motion was verified using a plantar pressure sensor or an electromyograph NPL 2 reports that a cross step is characterized by an increase in muscle activity of the gluteus medius muscle, tensor fascicularis femoris, gastrocnemius longus muscle, and lateral head of gastrocnemius muscle. NPL 2 reports that a side step is characterized by an increase in muscle activity of a plantarflexion/internalization muscle group (mainly the tibialis anterior muscle) and medial head of gastrocnemius muscle.

### CITATION LIST

### Patent Literature

PTL 1: WO 2021/140658 A
PTL 2: JP 2007-125368 A

### Non Patent Literature

NPL 1: Chihiro Kurosawa, “Kinematic analysis of healthy elder adults during Timed Up and Go test”, International University of Health and Welfare, Examination Dissertation (Doctorate), FY 2016.
NPL 2: Masanori Ito et al., “Change of direction while walking”, Kansai Physical Therapy Vol. 15, pp. 23-27, 2015.

### SUMMARY OF INVENTION

### Technical Problem

In the method of PTL 1, the progress state of hallux valgus is estimated using the gait feature amount of the feature portion extracted from the data acquired from the sensor installed in the footwear. PTL 1 does not disclose estimating the mobility using the gait feature amount of the feature portion extracted from the data acquired from the sensor installed on the footwear.

In the method of PTL 2, mobility of the subject is estimated according to the acceleration measured by the accelerometer installed on the waist of the subject. In the method of PTL 2, mobility such as gait speed, stride, knee extension force, and back bending force is estimated according to the calculated estimation index. In the method of PTL 2, the mobility of the subject is estimated according to the acceleration measured by the accelerometer of the waist. In the method of PTL 2, the mobility according to the movement of the waist is estimated, but the lower limb muscle strength according to the movement of the foot cannot be verified.

By evaluating TUG test as in NPL 1, standing and sitting, walking, and direction change included in the mobility can be evaluated in detail. As in NPL 2, if a plantar pressure sensor or an electromyograph is used, the direction change included in the mobility can be evaluated in detail. However, NPL 1 to 2 does not disclose a method for evaluating mobility in daily life.

An object of the present disclosure is to provide a mobility estimation device and the like capable of appropriately estimating a mobility in daily life.

### Solution to Problem

A mobility estimation device according to an aspect of the present disclosure includes a data acquisition unit that acquires feature amount data including a feature amount used for estimating a mobility of a user, the feature amount data being extracted from sensor data regarding a movement of a foot of the user, a storage unit that stores an estimation model that outputs a mobility index based on an input of the feature amount data, an estimation unit that inputs the acquired feature amount data to the estimation model and estimates the mobility of the user in accordance with the mobility index output from the estimation model, and an output unit that outputs information regarding the estimated mobility of the user.

A mobility estimating method according to one aspect of the present disclosure includes, by a computer, acquiring feature amount data including a feature amount used for estimating a mobility of a user, the feature amount data being extracted from sensor data regarding a movement of a foot of the user, inputting the acquired feature amount data to an estimation model that outputs a mobility index based on an input of the feature amount data, estimating the mobility of the user in accordance with the mobility index output from the estimation model, and outputting information regarding the estimated mobility of the user.

A program according to one aspect of the present disclosure causes a computer to execute processing of acquiring feature amount data including a feature amount used for estimating a mobility of a user, the feature amount data being extracted from sensor data regarding a movement of a foot of the user, processing of inputting the acquired feature amount data to an estimation model that outputs a mobility index based on an input of the feature amount data, processing of estimating the mobility of the user in accordance with the mobility index output from the estimation model, and processing of outputting information regarding the estimated mobility of the user.

### Advantageous Effects of Invention

According to the present disclosure, it is possible to provide a mobility estimation device and the like capable of appropriately estimating a mobility in daily life.

### BRIEF DESCRIPTION OF DRAWINGS

FIG. 1 is a block diagram illustrating an example of a configuration of a mobility estimation system according to a first example embodiment.

FIG. 2 is a block diagram illustrating an example of a configuration of a gait measuring device included in the mobility estimation system according to the first example embodiment.

FIG. 3 is a conceptual diagram illustrating an arrangement example of the gait measuring device according to the first example embodiment.

FIG. 4 is a conceptual diagram for describing an example of a relationship between a local coordinate system and a world coordinate system set in the gait measuring device according to the first example embodiment.

FIG. 5 is a conceptual diagram for describing a human body surface used in a description regarding the gait measuring device according to the first example embodiment.

FIG. 6 is a conceptual diagram for describing a gait cycle used in a description regarding the gait measuring device according to the first example embodiment.

FIG. 7 is a graph for describing an example of time-series data of sensor data measured by the gait measuring device according to the first example embodiment.

FIG. 8 is a diagram for describing an example of normalization of gait waveform data extracted from time-series data of sensor data measured by the gait measuring device according to the first example embodiment.

FIG. 9 is a conceptual diagram for describing an example of a gait phase cluster from which a feature amount data generating unit of the gait measuring device according to the first example embodiment extracts a feature amount.

FIG. 10 is a block diagram illustrating an example of a configuration of a mobility estimation device included in the mobility estimation system according to the first example embodiment.

FIG. 11 is a conceptual diagram for describing a TUG (Time Up and Go) test for evaluating a mobility to be estimated by the mobility estimation system according to the first example embodiment.

FIG. 12 is a table relating to specific examples of feature amounts extracted by the gait measuring device included in the mobility estimation system according to the first example embodiment in order to estimate a result of a TUG test (TUG required time).

FIG. 13 is a graph illustrating a correlation between a feature amount F1 extracted by the gait measuring device included in the mobility estimation system according to the first example embodiment and a measured TUG required time.

FIG. 14 is a graph illustrating a correlation between a feature amount F2 extracted by the gait measuring device included in the mobility estimation system according to the first example embodiment and a measured TUG required time.

FIG. 15 is a graph illustrating a correlation between a feature amount F3 extracted by the gait measuring device included in the mobility estimation system according to the first example embodiment and a measured TUG required time.

FIG. 16 is a graph illustrating a correlation between a feature amount F4 extracted by the gait measuring device included in the mobility estimation system according to the first example embodiment and a measured TUG required time.

FIG. 17 is a graph illustrating a correlation between a feature amount F5 extracted by the gait measuring device included in the mobility estimation system according to the first example embodiment and a measured TUG required time.

FIG. 18 is a graph illustrating a correlation between a feature amount F6 extracted by the gait measuring device included in the mobility estimation system according to the first example embodiment and a measured TUG required time.

FIG. 19 is a block diagram illustrating an example of estimation of a TUG required time (mobility index) by the mobility estimation device included in the mobility estimation system according to the first example embodiment.

FIG. 20 is a graph illustrating a correlation between an estimated value of a TUG required time estimated using an estimation model generated by machine learning with gender, age, height, weight, and gait speed as explanatory variables and a measured value of the TUG required time.

FIG. 21 is a graph illustrating a correlation between the estimated value of the TUG required time estimated by the mobility estimation device included in the mobility estimation system according to the first example embodiment and the measured value of the TUG required time.

FIG. 22 is a flowchart for describing an example of the operation of the gait measuring device included in the mobility estimation system according to the first example embodiment.

FIG. 23 is a flowchart for describing an example of an operation of the mobility estimation device included in the mobility estimation system according to the first example embodiment.

FIG. 24 is a conceptual diagram for describing an application example of the mobility estimation system according to the first example embodiment.

FIG. 25 is a block diagram illustrating an example of a configuration of a machine learning system according to a second example embodiment.

FIG. 26 is a block diagram illustrating an example of a configuration of a machine learning device included in a machine learning system according to the second example embodiment.

FIG. 27 is a conceptual diagram for describing an example of machine learning by a machine learning device included in a machine learning system according to the second example embodiment.

FIG. 28 is a block diagram illustrating an example of a configuration of a mobility estimation device according to a third example embodiment.

FIG. 29 is a block diagram illustrating an example of a hardware configuration that executes control and processing according to each example embodiment.

### EXAMPLE EMBODIMENTS

Hereinafter, example embodiments of the present invention will be described with reference to the drawings. However, although the example embodiments to be described below are technically preferably limited in order to carry out the present invention, the scope of the invention is not limited to the following. In all the drawings used in the following description of the example embodiment, the same reference numerals are given to similar parts unless there is a particular reason. In the following example embodiments, repeated description of similar configurations and operations may be omitted.

### First Example Embodiment

First, a mobility estimation system according to a first example embodiment will be described with reference to the drawings. The mobility estimation system of the present example embodiment measures sensor data regarding movement of the foot according to the gait of the user. The mobility estimation system of the present example embodiment estimates the mobility of the user using the measured sensor data.

In the present example embodiment, an example of estimating the performance of a time up and go (TUG) test as the mobility will be described. In the present example embodiment, the score of the TUG test is evaluated by the time (also referred to as TUG required time) from standing up from the chair, walking to the mark to change the direction, and sitting down again on the chair. The TUG required time is a grade value of the TUG test. The shorter the TUG required time, the higher the TUG test performance. The method of the present example embodiment can also be applied to a test result regarding mobility other than the TUG test.

### (Configuration)

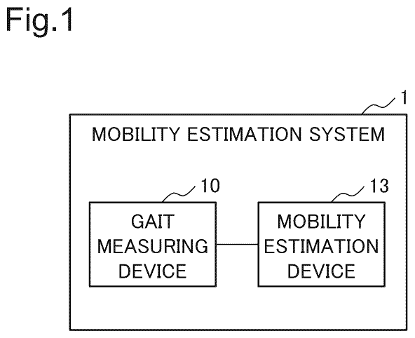

FIG. 1 is a block diagram illustrating an example of a configuration of a mobility estimation system 1 according to the present example embodiment. The mobility estimation system 1 includes a gait measuring device 10 and a mobility estimation device 13. In the present example embodiment, an example in which the gait measuring device 10 and the mobility estimation device 13 are configured as separate hardware will be described. For example, the gait measuring device 10 is installed on footwear or the like of a subject (user) who is an estimation target of the mobility. For example, the function of the mobility estimation device 13 is installed in a mobile terminal carried by a subject (user). Hereinafter, configurations of the gait measuring device 10 and the mobility estimation device 13 will be individually described.

### [Gait Measuring Device]

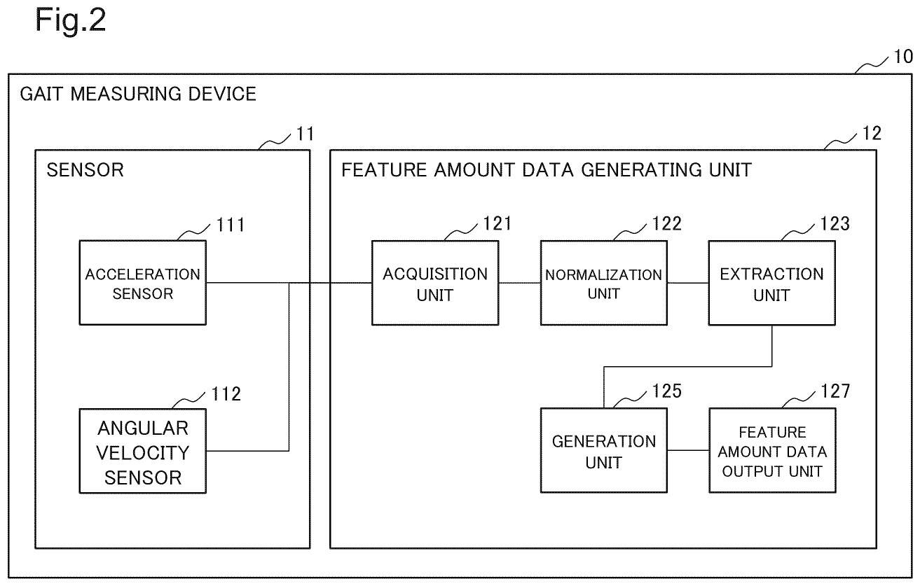

FIG. 2 is a block diagram illustrating an example of a configuration of the gait measuring device 10. The gait measuring device 10 includes a sensor 11 and a feature amount data generating unit 12. In the present example embodiment, an example in which the sensor 11 and the feature amount data generating unit 12 are integrated will be described. The sensor 11 and the feature amount data generating unit 12 may be provided as separate devices.

As illustrated in FIG. 2, the sensor 11 includes an acceleration sensor 111 and an angular velocity sensor 112. FIG. 2 illustrates an example in which the acceleration sensor 111 and the angular velocity sensor 112 are included in the sensor 11. The sensor 11 may include a sensor other than the acceleration sensor 111 and the angular velocity sensor 112. Sensors other than the acceleration sensor 111 and the angular velocity sensor 112 that can be included in the sensor 11 will not be described.

The acceleration sensor 111 is a sensor that measures an acceleration (also referred to as spatial accelerations) in three axial directions. The acceleration sensor 111 measures an acceleration (also referred to as spatial acceleration) as a physical quantity related to movement of the foot. The acceleration sensor 111 outputs measured acceleration to the feature amount data generating unit 12. For example, a sensor of a piezoelectric type, a piezoresistive type, a capacitance type, or the like can be used as the acceleration sensor 111. The sensor used as the acceleration sensor 111 is not limited to the measurement method as long as the sensor can measure acceleration.

The angular velocity sensor 112 is a sensor that measures an angular velocity (also referred to as a spatial angular velocity) around three axes. The angular velocity sensor 112 measures the angular velocity (also referred to as spatial angular velocity) as a physical quantity related to movement of the foot. The angular velocity sensor 112 outputs the measured angular velocity to the feature amount data generating unit 12. For example, a sensor of a vibration type, a capacitance type, or the like can be used as the angular velocity sensor 112. The sensor used as the angular velocity sensor 112 is not limited to the measurement method as long as the sensor can measure the angular velocity.

The sensor 11 is implemented by, for example, an inertial measuring device that measures acceleration and angular velocity. An example of the inertial measuring device is an inertial measurement unit (IMU). The IMU includes the acceleration sensor 111 that measures accelerations in three-axis directions and the angular velocity sensor 112 that measures angular velocities around the three axes. The sensor 11 may be implemented by an inertial measuring device such as a vertical gyro (VG) or an attitude heading (AHRS). The sensor 11 may be implemented by global positioning system/inertial navigation system (GPS/INS). The sensor 11 may be implemented by a device other than the inertial measuring device as long as it can measure a physical quantity related to movement of the foot.

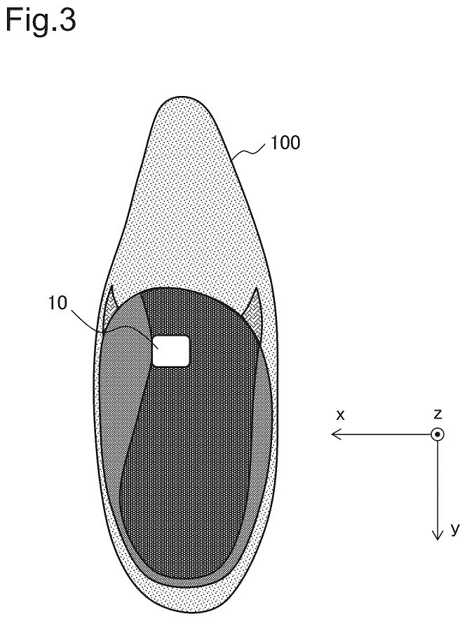

FIG. 3 is a conceptual diagram illustrating an example in which the gait measuring device 10 is arranged in a shoe 100 of the right foot. In the example of FIG. 3, the gait measuring device 10 is installed at a position corresponding to the back side of the arch of foot. For example, the gait measuring device 10 is arranged in an insole inserted into the shoe 100. For example, the gait measuring device 10 may be arranged on the bottom surface of the shoe 100. For example, the gait measuring device 10 may be embedded in the main body of the shoe 100. The gait measuring device 10 may be detachable from the shoe 100 or may not be detachable from the shoe 100. The gait measuring device 10 may be installed at a position other than a back side of the arch of foot as long as sensor data regarding the movement of the foot can be measured. The gait measuring device 10 may be installed on a sock worn by the user or a decorative article such as an anklet worn by the user. The gait measuring device 10 may be directly attached to the foot or may be embedded in the foot. FIG. 3 illustrates an example in which the gait measuring device 10 is installed in the shoe 100 of the right foot. The gait measuring device 10 may be installed on the shoes 100 of both feet.

In the example of FIG. 3, a local coordinate system including an x axis in the left-right direction, a y axis in the front-back direction, and a z axis in the up-down direction is set with reference to the gait measuring device 10 (sensor 11). In the x-axis, the left side is positive, in the y-axis, the rear side is positive, and in the z-axis, the upper side is positive. The direction of the axis set in the sensor 11 may be the same for the left and right feet, or may be different for the left and right feet. For example, in a case where the sensors 11 produced with the same specifications are arranged in the left and right shoes 100, the vertical directions (directions in the Z-axis direction) of the sensors 11 arranged in the left and right shoes 100 are the same. In this case, the three axes of the local coordinate system set in sensor data derived from the left foot and the three axes of the local coordinate system set in sensor data derived from the right foot are the same on the left and right.

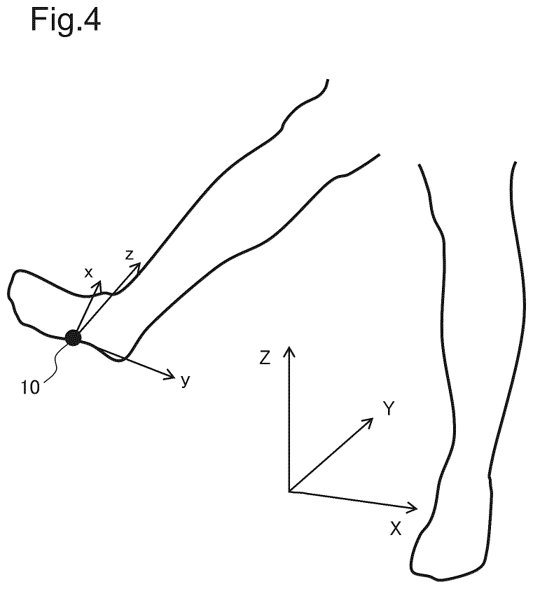

FIG. 4 is a conceptual diagram for describing a local coordinate system (x-axis, y-axis, z-axis) set in the gait measuring device 10 (sensor 11) installed on the back side of the arch of foot and a world coordinate system (X axis, Y axis, Z axis) set with respect to the ground. In the world coordinate system (X axis, Y axis, Z axis), in a state where the user facing a traveling direction is upright, a lateral direction of the user is set to the X-axis direction (a leftward direction is positive), a direction of the back surface of the user is set to the Y-axis direction (a rearward direction is positive), and a gravity direction is set to the Z-axis direction (a vertically upward direction is positive). The example of FIG. 4 conceptually illustrates the relationship between the local coordinate system (x-axis, y-axis, z-axis) and the world coordinate system (X axis, Y axis, Z axis), and does not accurately illustrate the relationship between the local coordinate system and the world coordinate system that varies depending on the gait of the user.

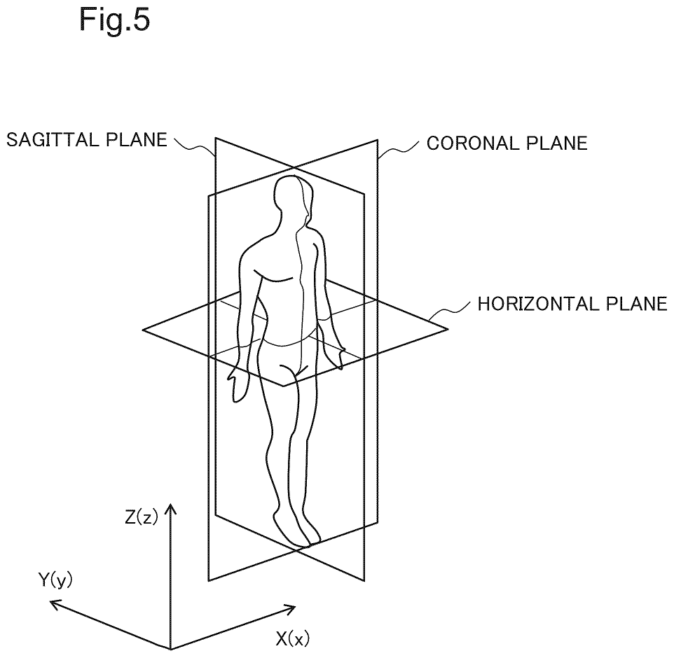

FIG. 5 is a conceptual diagram for describing a surface (also referred to as a human body surface) set for the human body. In the present example embodiment, a sagittal plane dividing the body into left and right, a coronal plane dividing the body into front and rear, and a horizontal plane dividing the body horizontally are defined. As illustrated in FIG. 5, the world coordinate system and the local coordinate system coincide with each other in a state in which a center line of the foot is oriented in the traveling direction. In the present example embodiment, rotation in the sagittal plane with the x-axis as the rotation axis is defined as roll, rotation in the coronal plane with the y-axis as the rotation axis is defined as pitch, and rotation in the horizontal plane with the z-axis as the rotation axis is defined as yaw. A rotation angle in the sagittal plane with the x axis as a rotation axis is defined as a roll angle, a rotation angle in the coronal plane with the y axis as a rotation axis is defined as a pitch angle, and a rotation angle in the horizontal plane with the z axis as a rotation axis is defined as a yaw angle.

As illustrated in FIG. 2, the feature amount data generating unit 12 (also referred to as a feature amount data generation device) includes an acquisition unit 121, a normalization unit 122, an extraction unit 123, a generation unit 125, and a feature amount data output unit 127. For example, the feature amount data generating unit 12 is implemented by a microcomputer or a microcontroller that performs overall control and data processing of the gait measuring device 10. For example, the feature amount data generating unit 12 includes a central processing unit (CPU), a random access memory (RAM), a read only memory (ROM), a flash memory, and the like. The feature amount data generating unit 12 controls the acceleration sensor 111 and the angular velocity sensor 112 to measure the angular velocity and the acceleration. For example, the feature amount data generating unit 12 may be implemented on a mobile terminal (not illustrated) carried by a subject (user).

The acquisition unit 121 acquires accelerations in three axial directions from the acceleration sensor 111. The acquisition unit 121 acquires angular velocities around three axes from the angular velocity sensor 112. For example, the acquisition unit 121 performs analog-to-digital conversion (AD conversion) on acquired physical quantities (analog data) such as angular velocity and acceleration. The physical quantities (analog data) measured by the acceleration sensor 111 and the angular velocity sensor 112 may be converted into digital data in each of the acceleration sensor 111 and the angular velocity sensor 112. The acquisition unit 121 outputs the converted digital data (also referred to as sensor data) to the normalization unit 122. The acquisition unit 121 may be configured to store the sensor data in a storage unit (not illustrated). The sensor data includes at least acceleration data converted into digital data and angular velocity data converted into digital data. The acceleration data includes acceleration vectors in three axial directions. The angular velocity data includes angular velocity vectors around three axes. The acceleration data and the angular velocity data are associated with acquisition times of the data. The acquisition unit 121 may add a correction such as a mounting error, a temperature correction, or a linearity correction to the acceleration data and the angular velocity data.

The normalization unit 122 acquires sensor data from the acquisition unit 121. The normalization unit 122 extracts time-series data (also referred to as gait waveform data) for one gait cycle from the time-series data of the accelerations in the three-axis directions and the angular velocities around the three axes included in the sensor data. The normalization unit 122 normalizes (also referred to as first normalization) time of the extracted gait waveform data for one gait cycle to a gait cycle of 0 to 100% (percent). Timing such as 1% or 10% included in the gait cycle of 0 to 100% is also referred to as a gait phase. The normalization unit 122 normalizes (also referred to as second normalization) gait waveform data for one gait cycle having subjected to the first normalization in such a way that a stance phase is 60% and a swing phase is 40%. The stance phase is a period in which at least a part of the back side of the foot is in contact with the ground. The swing phase is a period in which the back side of the foot is away from the ground. When the gait waveform data is subjected to the second normalization, it is possible to suppress deviation of the gait phase from which a feature amount is extracted from fluctuating due to the influence of disturbance.

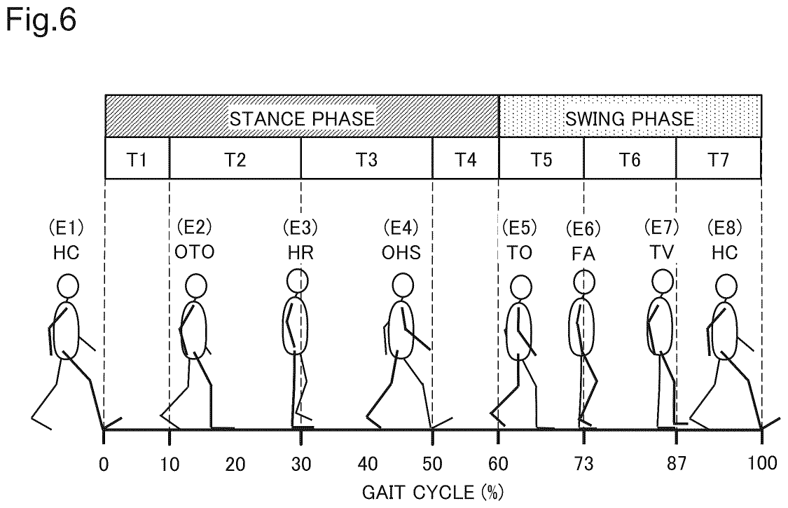

FIG. 6 is a conceptual diagram for describing one gait cycle with the right foot as a reference. One gait cycle based on the left foot is also similar to that of the right foot. The horizontal axis of FIG. 6 is one gait cycle of the right foot with a time point at which the heel of the right foot lands on the ground as a starting point and a time point at which the heel of the right foot next lands on the ground as an ending point. The horizontal axis in FIG. 6 has been subjected to the first normalization with one gait cycle as 100%. In the horizontal axis of FIG. 6, the second normalization is performed in such a way that the stance phase is 60% and the swing phase is 40%. In general, one gait cycle of one foot is roughly divided into a stance phase in which at least a part of the back side of the foot is in contact with the ground and a swing phase in which the back side of the foot is away from the ground. The stance phase is further subdivided into a load response period T1, a mid-stance period T2, a terminal stance period T3, and a pre-swing period T4. The swing phase is further subdivided into an initial swing period T5, a mid-swing period T6, and a terminal swing period T7. FIG. 6 is an example, and does not limit the periods constituting one gait cycle, the names of these periods, and the like.

As illustrated in FIG. 6, in a gait, multiple events (also referred to as gait events) occur. E1 represents an event in which the heel of the right foot touches the ground (heel contact (HC)). E2 represents an event in which the toe of the left foot is separated from the ground with the sole of the right foot in contact with the ground (opposite toe off (OTO)). E3 represents an event in which the heel of the right foot rises with the sole of the right foot in contact with the ground (heel rise (HR)). E4 is an event in which the heel of the left foot touches the ground (opposite heel strike (OHS)). E5 represents an event in which the toe of the right foot is separated from the ground with the sole of the left foot in contact with the ground (toe off (TO)). E6 represents an event in which the left foot and the right foot cross with the sole of the left foot in contact with the ground (foot adjacent (FA)). E7 represents an event in which the tibia of the right foot is approximately perpendicular to the ground with the sole of the left foot in contact with the ground (tibia vertical (TV)). E8 represents an event in which the heel of the right foot touches the ground (heel contact (HC)). E8 corresponds to the end point of the gait cycle starting from E1 and corresponds to the start point of the next gait cycle. FIG. 6 is an example, and does not limit events that occur during a gait or names of these events.

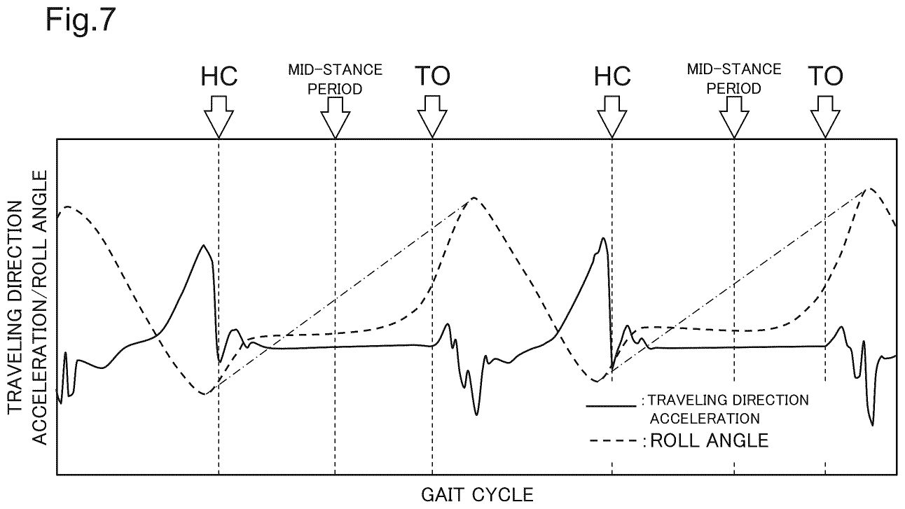

FIG. 7 is a diagram for describing an example of detecting the heel contact HC and the toe off TO from time-series data (solid line) of a traveling direction acceleration (Y-direction acceleration). The timing of the heel contact HC is a timing of a minimum peak immediately after a maximum peak appearing in the time-series data of the traveling direction acceleration (Y-direction acceleration). A maximum peak serving as a mark of the timing of the heel contact HC corresponds to a largest peak of gait waveform data for one gait cycle. A section between consecutive heel contact HC is one gait cycle. The timing of the toe off TO is a rising timing of a maximum peak appearing after the period of the stance phase in which fluctuation does not appear in the time-series data of the traveling direction acceleration (Y-direction acceleration). FIG. 7 also illustrates time-series data (broken line) of a roll angle (angular velocity around the X axis). A timing at a midpoint between a timing at which the roll angle is minimum and a timing at which the roll angle is maximum corresponds to the mid-stance period. For example, parameters such as gait speed, stride, circumduction, medial/lateral rotation, and plantarflexion/dorsiflexion (also referred to as gait parameters) can be obtained with reference to the mid-stance period.

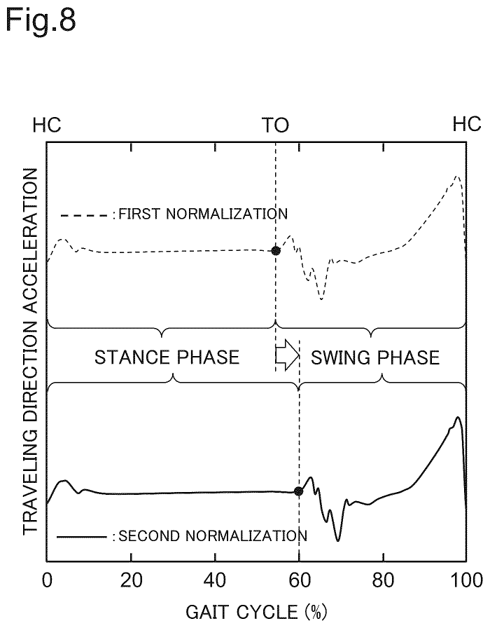

FIG. 8 is a diagram for describing an example of the gait waveform data normalized by the normalization unit 122. The normalization unit 122 detects the heel contact HC and the toe off TO from the time-series data of the traveling direction acceleration (Y-direction acceleration). The normalization unit 122 extracts a section between consecutive heel contacts HC as gait waveform data for one gait cycle. The normalization unit 122 converts the horizontal axis (time axis) of the gait waveform data for one gait cycle into a gait cycle of 0 to 100% by the first normalization. In FIG. 8, the gait waveform data after the first normalization is indicated by a broken line. In the gait waveform data (broken line) after the first normalization, the timing of the toe off TO deviates from 60%.

In the example of FIG. 8, the normalization unit 122 normalizes a section from the heel contact HC at which the gait phase is 0% to the toe off TO subsequent to the heel contact HC to 0 to 60%. The normalization unit 122 normalizes a section from the toe off TO to the heel contact HC at which the gait phase subsequent to the toe off TO is 100% to 60 to 100%. As a result, the gait waveform data for one gait cycle is normalized to a section (stance phase) in which the gait cycle is 0 to 60% and a section (swing phase) in which the gait cycle is 60 to 100%. In FIG. 8, the gait waveform data after the second normalization is indicated by a solid line. In the gait waveform data (solid line) after the second normalization, the timing of the toe off TO coincides with 60%.

FIGS. 7 to 8 illustrate examples in which the gait waveform data for one gait cycle is extracted/normalized based on the traveling direction acceleration (Y-direction acceleration). With respect to acceleration/angular velocity other than the traveling direction acceleration (Y-direction acceleration), the normalization unit 122 extracts/normalizes gait waveform data for one gait cycle in accordance with the gait cycle of the traveling direction acceleration (Y-direction acceleration). The normalization unit 122 may generate time-series data of angles around three axes by integrating time-series data of angular velocities around the three axes. In this case, the normalization unit 122 also extracts/normalizes the gait waveform data for one gait cycle in accordance with the gait cycle of the traveling direction acceleration (Y-direction acceleration) with respect to the angle around the three axes.

The normalization unit 122 may extract/normalize the gait waveform data for one gait cycle based on acceleration/angular velocity other than the traveling direction acceleration (Y-direction acceleration) (drawings are omitted). For example, the normalization unit 122 may detect the heel contact HC and the toe off TO from time-series data of vertical acceleration (Z-direction acceleration). The timing of the heel contact HC is a timing of a steep minimum peak appearing in the time-series data of the vertical acceleration (Z-direction acceleration). At the timing of the steep minimum peak, the value of the vertical acceleration (Z-direction acceleration) becomes substantially zero. The minimum peak serving as a mark of the timing of the heel contact HC corresponds to a smallest peak of the gait waveform data for one gait cycle. A section between consecutive heel contact HC is one gait cycle. The timing of the toe off TO is a timing of an inflection point in the middle of gradually increasing after the time-series data of the vertical acceleration (Z-direction acceleration) passes through a section with a small fluctuation after the maximum peak immediately after the heel contact HC. The normalization unit 122 may extract/normalize the gait waveform data for one gait cycle based on both the traveling direction acceleration (Y-direction acceleration) and the vertical acceleration (Z-direction acceleration). The normalization unit 122 may extract/normalize the gait waveform data for one gait cycle based on acceleration, angular velocity, angle, and the like other than the traveling direction acceleration (Y-direction acceleration) and the vertical acceleration (Z-direction acceleration).

The extraction unit 123 acquires the gait waveform data for one gait cycle normalized by the normalization unit 122. The extraction unit 123 extracts a feature amount used for estimating mobility from the gait waveform data for one gait cycle. The extraction unit 123 extracts a feature amount for each gait phase cluster from a gait phase cluster obtained by integrating temporally continuous gait phases based on a preset condition. The gait phase cluster includes at least one gait phase. The gait phase cluster also includes a single gait phase. The gait waveform data and the gait phase from which the feature amount used for estimating mobility is extracted will be described later.

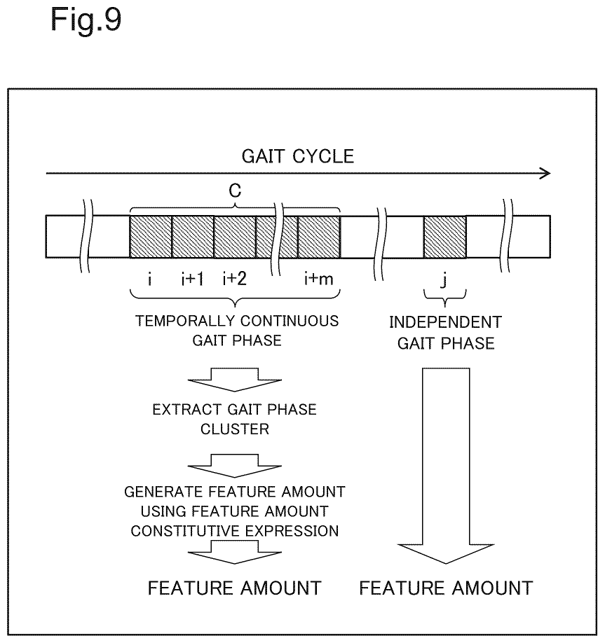

FIG. 9 is a conceptual diagram for describing extraction of a feature amount for estimating mobility from gait waveform data for one gait cycle. For example, the extraction unit 123 extracts temporally continuous gait phases i to i+m as the gait phase cluster C (i and m are natural numbers). The gait phase cluster C includes m gait phases (components). That is, the number of gait phases (components) (also referred to as the number of components) constituting the gait phase cluster C is m. FIG. 9 illustrates an example in which the gait phase has an integer value, but the gait phase may be subdivided into decimal places. When the gait phase is subdivided into decimal places, the number of components of the gait phase cluster C is a number corresponding to the number of data points in the section of the gait phase cluster. The extraction unit 123 extracts a feature amount from each of the gait phases i to i+m. In a case where the gait phase cluster C includes a single gait phase j, the extraction unit 123 extracts a feature amount from the single gait phase j (j is a natural number).

The generation unit 125 applies a feature amount constitutive expression to the feature amount (first feature amount) extracted from each of the gait phases constituting the gait phase cluster to generate a feature amount (second feature amount) of the gait phase cluster. The feature amount constitutive expression is a preset calculation expression for generating a feature amount of a gait phase cluster. For example, the feature amount constitutive expression is a calculation expression related to four arithmetic operations. For example, the second feature amount calculated using the feature amount constitutive expression is an integral average value, an arithmetic average value, an inclination, a variation, or the like of the first feature amount in each gait phase included in the gait phase cluster. For example, the generation unit 125 applies a calculation expression for calculating the inclination or variation of the first feature amount extracted from each of the gait phases constituting the gait phase cluster as the feature amount constitutive expression. For example, in a case where the gait phase cluster is configured by an independent gait phase, it is not possible to calculate the inclination or variation, and thus it is sufficient to use a feature amount constitutive expression for calculating an integral average value, an arithmetic average value, or the like.

The feature amount data output unit 127 outputs the feature amount data for each gait phase cluster generated by the generation unit 125. The feature amount data output unit 127 outputs the generated feature amount data of the gait phase cluster to the mobility estimation device 13 that uses the feature amount data.

### [Mobility Estimation Device]

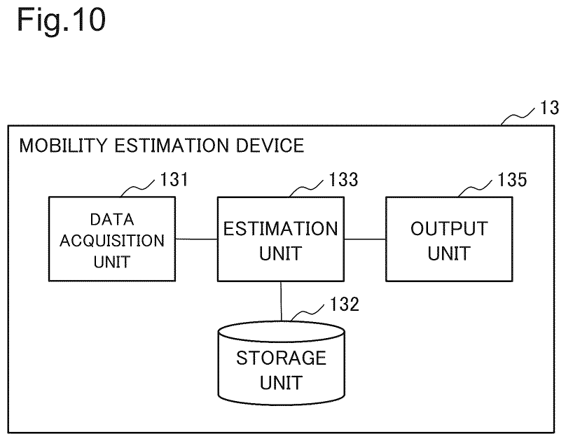

FIG. 10 is a block diagram illustrating an example of a configuration of the mobility estimation device 13. The mobility estimation device 13 includes a data acquisition unit 131, a storage unit 132, an estimation unit 133, and an output unit 135.

The data acquisition unit 131 acquires feature amount data from the gait measuring device 10. The data acquisition unit 131 outputs the received feature amount data to the estimation unit 133. The data acquisition unit 131 may receive the feature amount data from the gait measuring device 10 via a wire such as a cable, or may receive the feature amount data from the gait measuring device 10 via wireless communication. For example, the data acquisition unit 131 is configured to receive the feature amount data from the gait measuring device 10 via a wireless communication function (not illustrated) conforming to a standard such as Bluetooth (registered trademark) or WiFi (registered trademark). The communication function of the data acquisition unit 131 may conform to a standard other than Bluetooth (registered trademark) or WiFi (registered trademark).

The storage unit 132 stores an estimation model for estimating the TUG required time as the mobility index using the feature amount data extracted from the gait waveform data. The storage unit 132 stores an estimation model that has machine-learned the relationship between the feature amount data related to the TUG required times of the plurality of subjects and the TUG required time. For example, the storage unit 132 stores an estimation model for estimating the TUG required time learned for a plurality of subjects. The TUG required time is affected by age. Thus, the storage unit 132 may store an estimation model according to attribute data regarding age.

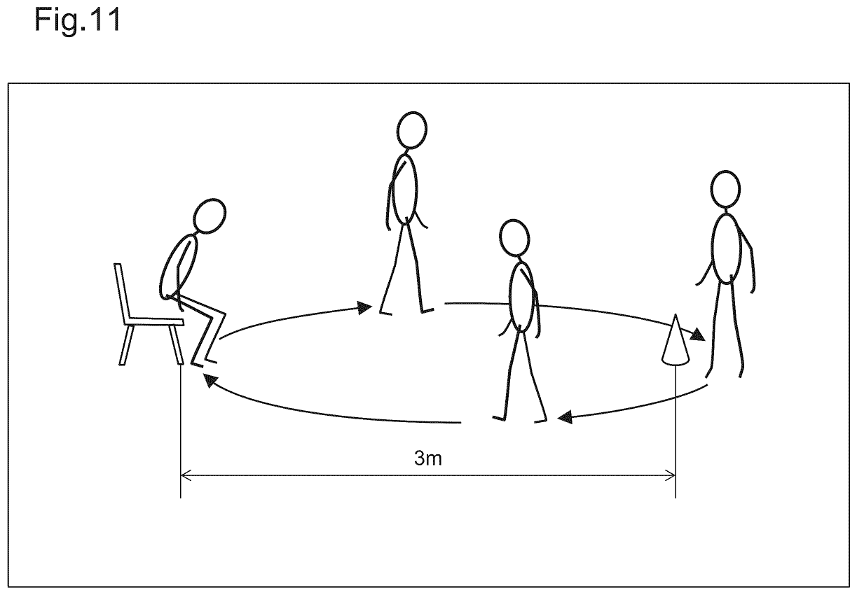

FIG. 11 is a conceptual diagram for describing the TUG test. The subject stands up from a state of sitting on a chair and walks toward the position of a mark. When the subject reaches the position of the mark, the subject changes the direction at the position of the mark and walks toward the chair on which the subject has just sat. When the subject returns to the chair, the subject sits on the chair. Measurement is started from the time point at which the user stands up from the chair, turns back at the mark, and the measurement is ended at the time point at which the user sits down again on the chair. The time required for this series of operations is the TUG required time.

The mobility can be evaluated according to the TUG required time. According to NPL 1, when the TUG required time is 7.4 seconds or more for men and 7.5 seconds or more for women, it corresponds to a specific elderly person (NPL 1: Chihiro Kurosawa, “Kinematic analysis of healthy elder adults during Timed Up and Go Test”, International University of Health and Welfare, Examination Dissertation (Doctorate), FY 2016). The evaluation criteria of the mobility according to the TUG required time described herein is a guide, and only needs to be set according to the situation.

The estimation model only needs to be stored in the storage unit 132 at the time of factory shipment of a product, calibration before the user uses the mobility estimation system 1, or the like. For example, an estimation model stored in a storage device such as an external server may be used. In that case, the estimation model only needs to be configured to be used via an interface (not illustrated) connected to the storage device.

The estimation unit 133 acquires the feature amount data from the data acquisition unit 131. The estimation unit 133 estimates the TUG required time as the mobility using the acquired feature amount data. The estimation unit 133 inputs the feature amount data to the estimation model stored in the storage unit 132. The estimation unit 133 outputs an estimation result corresponding to the mobility (TUG required time) output from the estimation model. In a case where an estimation model stored in an external storage device constructed in a cloud, a server, or the like is used, the estimation unit 133 is configured to use the estimation model via an interface (not illustrated) connected to the storage device.

The output unit 135 outputs the estimation result of the mobility by the estimation unit 133. For example, the output unit 135 displays the estimation result of the mobility on the screen of the mobile terminal of the subject (user). For example, the output unit 135 outputs the estimation result to an external system or the like that uses the estimation result. Use of the mobility output from the mobility estimation device 13 is not particularly limited.

For example, the mobility estimation device 13 is connected to an external system or the like constructed in a cloud or a server via a mobile terminal (not illustrated) carried by a subject (user). The mobile terminal (not illustrated) is a portable communication device. For example, the mobile terminal is a portable communication device having a communication function, such as a smartphone, a smart watch, or a mobile phone. For example, the mobility estimation device 13 is connected to the mobile terminal via a wire such as a cable. For example, the mobility estimation device 13 is connected to the mobile terminal via wireless communication. For example, the mobility estimation device 13 is connected to the mobile terminal via a wireless communication function (not illustrated) conforming to a standard such as Bluetooth (registered trademark) or WiFi (registered trademark). The communication function of the mobility estimation device 13 may conform to a standard other than Bluetooth (registered trademark) or WiFi (registered trademark). The estimation result of the mobility may be used by an application installed on the mobile terminal. In that case, the mobile terminal executes processing using the estimation result by application software or the like installed in the mobile terminal.

### [Estimation of TUG Required Time]

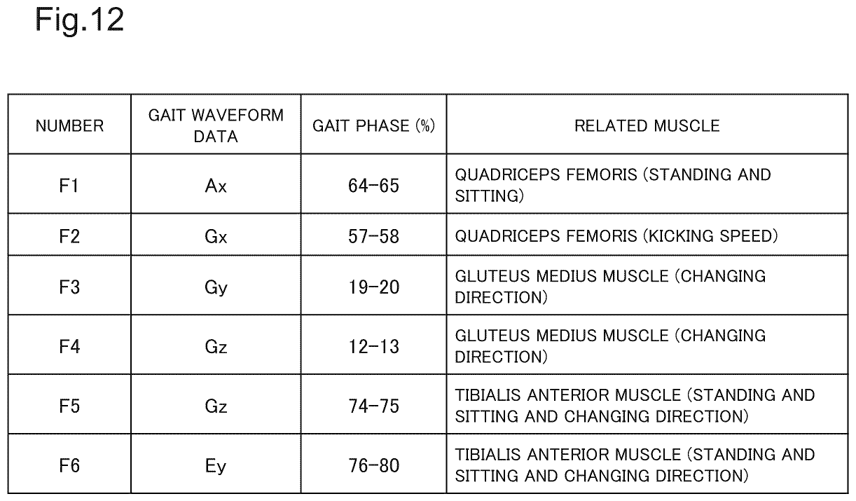

Next, the correlation between the TUG required time and the feature amount data will be described with reference to a verification example. FIG. 12 is a correspondence table summarizing feature amounts used for estimating the TUG required time. The correspondence table of FIG. 12 associates the number of the feature amount, the gait waveform data from which the feature amount is extracted, the gait phase (%) from which the gait phase cluster is extracted, and the related muscle. The TUG required time is correlated with the quadriceps femoris, the gluteus medius muscle, and the tibialis anterior muscle. Thus, feature amounts F1 to F5 extracted from the gait phase in which these features appear are used to estimate the TUG required time.

The TUG test includes three parts: standing, sitting, walking, and changing direction. The standing and sitting is mainly related to the tibialis anterior muscle, gastrocnemius muscle, quadriceps femoris, and biceps femoris. Related to gait performance are mainly stride length, gait speed, and cadence. The cadence is the number of steps per minute. The changing direction is related to the muscles used in the cross step and the side step. Muscles related to cross steps and side steps are disclosed in NPL 2 (NPL 2: Masaaki Ito et al., “Change of direction while walking”, Kansai Physical Therapy, Vol. 15, pp. 23-27, 2015). The cross step is associated with the gluteus medius muscle, tensor fascicularis femoris, gastrocnemius longus muscle, and lateral head of gastrocnemius muscle. The side step is associated with a plantarflexion/internalization muscle group (mainly the tibialis anterior muscle) and medial head of gastrocnemius muscle. Features of muscles related to changing direction appear in a specific gait phase. Features of the gluteus medius muscle appear in the gait phase 0 to 25%. Features of the tensor fascicularis femoris appear in the gait phases 0 to 45% and 85 to 100%. Features of the gastrocnemius longus muscle appear in the gait phase 10 to 50%. Features of the tibialis anterior muscle appear in the gait phases 0 to 10% and 57 to 100%. Features of the gastrocnemius muscle appear in the gait phase 10 to 50%.

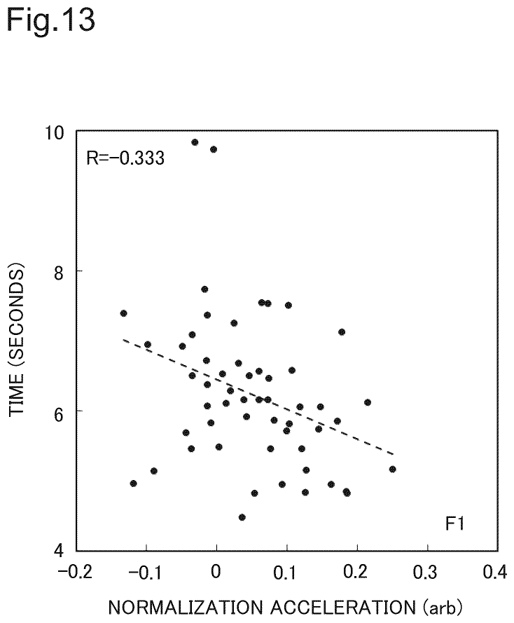

FIGS. 13 to 18 are verification results of the correlation between the TUG required time and the feature amount data. FIGS. 13 to 18 illustrate results of verification performed on a total of 62 subjects including 27 males and 35 females aged 60 to 85 years. FIGS. 13 to 18 illustrate results of verifying the correlation between estimated values estimated using feature amounts extracted in accordance with a gait while wearing footwear equipped with the gait measuring device 10 and measured values (true values) of the TUG required time.

The feature amount F1 is extracted from a section of a gait phase 64 to 65% of gait waveform data Ax related to time-series data of the lateral acceleration (X-direction acceleration). The gait phase 64 to 65% is included in the initial swing period T5. The feature amount F1 mainly includes a feature related to movement of the quadriceps femoris in the standing and sitting motion. FIG. 13 is a verification result of the correlation between the feature amount F1 and the TUG required time. The horizontal axis of the graph of FIG. 13 is a normalized angular velocity. The correlation coefficient R between the feature amount F1 and the TUG required time was −0.333.

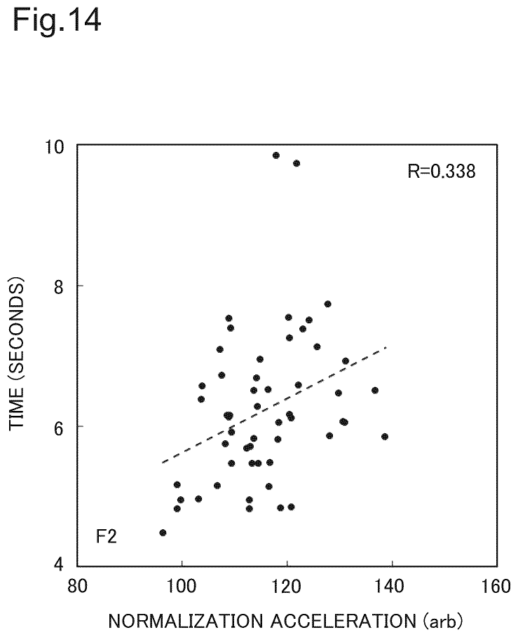

The feature amount F2 is extracted from a section of a gait phase 57 to 58% of gait waveform data Gx related to the time-series data of the angular velocity in the sagittal plane (around the X axis). The gait phase 57 to 58% is included in the pre-swing period T4. The feature amount F2 mainly includes a feature related to the motion of the quadriceps femoris related to a leg kicking speed. FIG. 14 is a verification result of the correlation between the feature amount F2 and the TUG required time. The horizontal axis of the graph of FIG. 1 is a normalized angular velocity. The correlation coefficient R between the feature amount F2 and the TUG required time was 0.338.

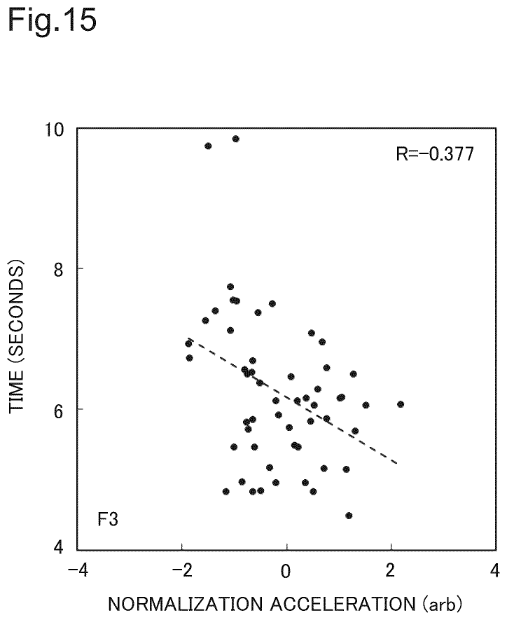

The feature amount F3 is extracted from a section of the gait phase 19 to 20% of the gait waveform data Gy related to the time-series data of the angular velocity in the coronal plane (around the Y axis). The gait phase 19 to 20% is included in the mid-stance period T2. The feature amount F3 mainly includes a feature related to movement of the gluteus medius muscle in the direction change. FIG. 15 is a verification result of the correlation between the feature amount F3 and the TUG required time. The horizontal axis of the graph of FIG. 15 is a normalized angular velocity. The correlation coefficient R between the feature amount F3 and the TUG required time was −0.377.

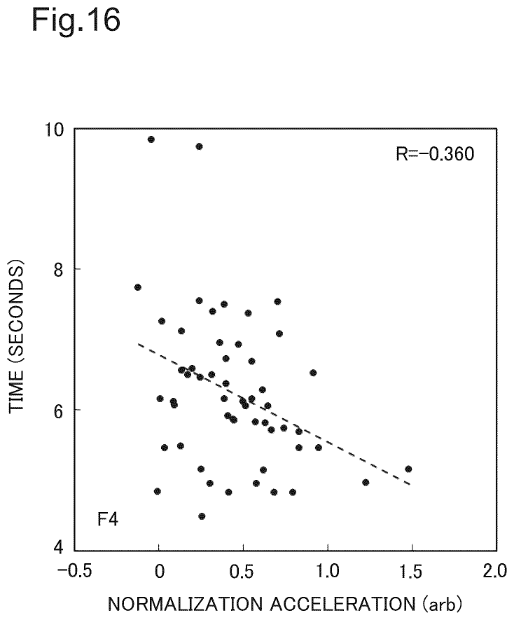

The feature amount F4 is extracted from the section of the gait phase 12 to 13% of the gait waveform data Ez related to the time-series data of the angular velocity in the horizontal plane (around the Z axis). The gait phase 12 to 13% is an early stage of the mid-stance period T2. The feature amount F4 mainly includes a feature related to movement of the gluteus medius muscle in the direction change. FIG. 16 is a verification result of the correlation between the feature amount F4 and the TUG required time. The horizontal axis of the graph of FIG. 16 is a normalized angular velocity. The correlation coefficient R between the feature amount F4 and the TUG required time was −0.360.

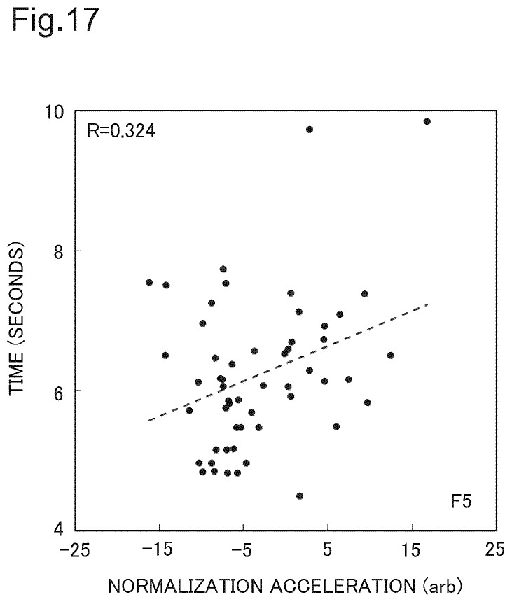

The feature amount F5 is extracted from the section of the gait phase 74 to 75% of the gait waveform data Ez related to the time-series data of the angular velocity in the horizontal plane (around the Z axis). The gait phase 74 to 75% is an early stage of the mid-swing period T6. The feature amount F5 mainly includes a feature related to movement of the tibialis anterior muscle in standing and sitting and changing direction. FIG. 17 is a verification result of the correlation between the feature amount F5 and the TUG required time. The horizontal axis of the graph of FIG. 17 is a normalized angular velocity. The correlation coefficient R between the feature amount F5 and the TUG required time was 0.324.

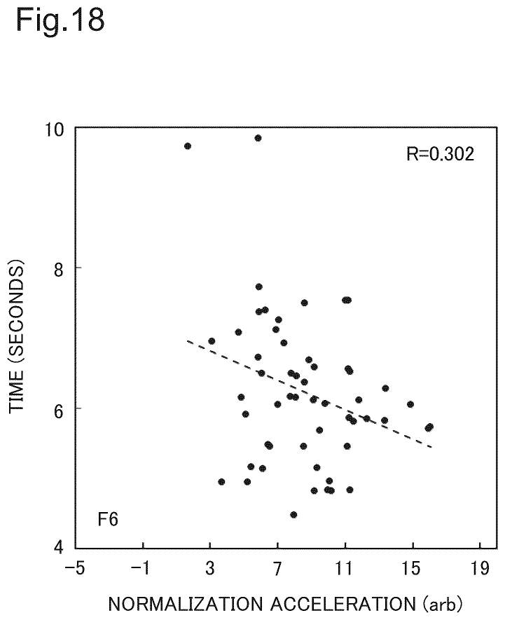

The feature amount F6 is extracted from a section of the gait phase 76 to 80% of the gait waveform data Ey related to the time-series data of the angle (posture angle) in the coronal plane (around the Y axis). The gait phase 76 to 80% is included in the mid-swing period T6. The feature amount F6 mainly includes a feature related to movement of the tibialis anterior muscle in standing and sitting and changing direction. FIG. 18 is a verification result of the correlation between the feature amount F6 and the TUG required time. The horizontal axis of the graph of FIG. 18 is an angle in the coronal plane (around the Y axis). The correlation coefficient R between the feature amount F6 and the TUG required time was 0.302.

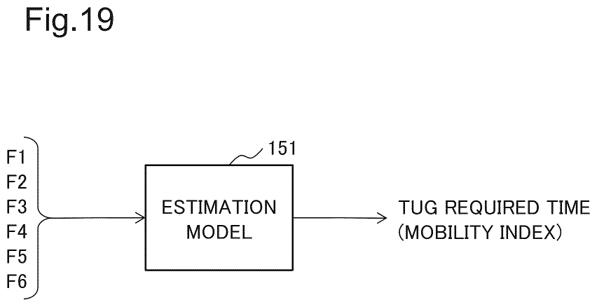

FIG. 19 is a conceptual diagram illustrating an example of inputting the feature amounts F1 to F6 extracted from the sensor data measured along with a gait of the user to the estimation model 151 constructed in advance to estimate the TUG required time as the mobility. The estimation model 151 outputs the TUG required time, which is a mobility index, according to the inputs of the feature amounts F1 to F6. For example, the estimation model 151 is generated by machine learning using teacher data having the feature amounts F1 to F6 used for estimating the TUG required time as explanatory variables and the TUG required time as an objective variable. The estimation result of the estimation model 151 is not limited as long as the estimation result regarding the TUG required time, which is an index of the mobility, is output in response to the input of the feature amount data for estimating the TUG required time. For example, the estimation model 151 may be a model that estimates the TUG required time using attribute data (age) as an explanatory variable in addition to the feature amounts F1 to F6 used for estimating the TUG required time.

For example, the storage unit 132 stores an estimation model for estimating the TUG required time using a multiple regression prediction method. For example, the storage unit 132 stores a parameter for estimating the TUG required time T using the following Expression 1.

$$T = a 1 \times F 1 + a 2 \times F 2 + a 3 \times F 3 + a 4 \times F 4 + a 5 \times f 5 + a 6 \times F 6 + a 0 \qquad ( 1 )$$

In Expression 1 described above, F1, F2, F3, F4, F5, and F6 are feature amounts for each gait phase cluster used for estimating the TUG required time illustrated in the correspondence table in FIG. 12. a1, a2, a3, a4, a5, and a6 are coefficients multiplied by F1, F2, F3, F4, F5, and F6. a0 is a constant term. For example, a0, a1, a2, a3, a4, a5, and a6 are stored in the storage unit 132.

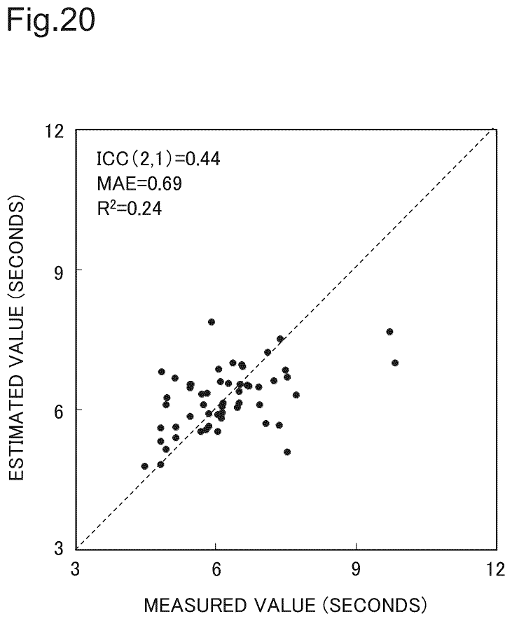

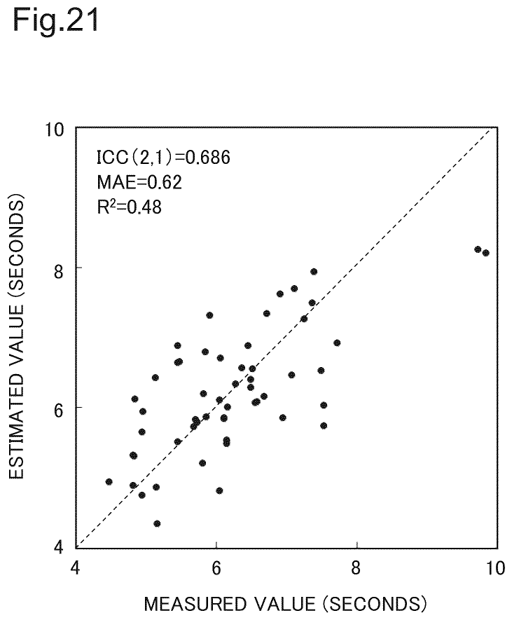

Next, a result of evaluating the estimation model 151 generated using the measurement data of the 62 subjects described above will be described. Here, a verification example (FIG. 20) in which the mobility (TUG required time) is estimated using attributes (including the gait speed) of the subject is compared with a verification example (FIG. 21) in which the mobility (TUG required time) is estimated using feature amounts of a gait of the subject. FIGS. 20 and 21 illustrate results of testing the estimation model generated using the measurement data of 61 people using the measurement data of the remaining 1 person by the LOSO (Leave-One-Subject-Out) method. FIGS. 20 and 21 illustrate results of performing LOSO on all (62) subjects and associating prediction values by the test with measured values (true value). The test result of LOSO was evaluated by values of intraclass correlation coefficients (ICC), a mean absolute error (MAE), and a determination coefficient R². As the intraclass correlation coefficient ICC, an intraclass correlation coefficient ICC (2, 1) was used in order to evaluate inter-examiner reliability.

FIG. 20 illustrates a verification result of an estimation model of a comparative example in which teacher data is machine-learned with gender, age, height, weight, and gait speed as explanatory variables and the TUG required time as an objective variable. In the estimation model of the comparative example, the intraclass correlation coefficient ICC (2, 1) was 0.44, the mean absolute error MAE was 0.69, and the determination coefficient R² was 0.24. In the verification result of FIG. 20, the gait speed that greatly affects a gait accounting for 70% of the operation in the TUG test may be included in the explanatory variable, and the intraclass correlation coefficient ICC (2, 1) is somewhat high.

FIG. 21 illustrates a verification result of the estimation model 151 of the present example embodiment learned from teacher data in which the feature amounts F1 to F6 and the ages are set as explanatory variables and the TUG required time is set as an objective variable. In the estimation model 151 of the present example embodiment, the intraclass correlation coefficient ICC (2, 1) was 0.686, the mean absolute error MAE was 0.62, and the determination coefficient R² was 0.48. That is, the estimation model 151 of the present example embodiment has higher reliability and smaller error than the estimation model of the comparative example, and the objective variable is sufficiently described by the explanatory variables. That is, according to the method of the present example embodiment, it is possible to generate the estimation model 151 that is highly reliable, has a small error, and has the objective variable sufficiently described by the explanatory variables, as compared with the estimation model using only the attributes and the gait speed.

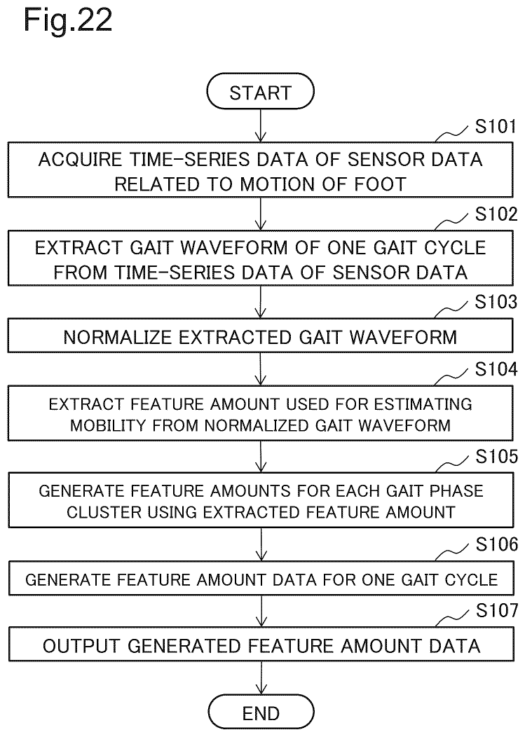

In the verification result of FIG. 22, the gait speed that greatly affects the gait occupying 70% of the operation in the TUG test is not included in the explanatory variables. However, the intraclass correlation coefficient ICC (2, 1) is higher in the verification result of FIG. 22 in which the gait speed is not used as an explanatory variable than in the verification result of FIG. 20 in which the gait speed is used as an explanatory variable. The feature amounts F1 to F6 may include the influence of the gait speed, but 30% of the motion in the TUG test is standing and sitting or changing direction. That is, results of the TUG test largely reflect not only the gait but also the influence of movement such as standing and sitting or changing direction. In other words, the gait speed is an important factor in the performance of the TUG test, but the performance of the TUG test cannot be estimated with high accuracy unless there is a feature amount expressing standing and sitting and changing direction.

### (Operation)

Next, an operation of the mobility estimation system 1 will be described with reference to the drawings. Here, the gait measuring device 10 and the mobility estimation device 13 included in the mobility estimation system 1 will be individually described. With respect to the gait measuring device 10, an operation of the feature amount data generating unit 12 included in the gait measuring device 10 will be described.

### [Gait Measuring Device]

FIG. 22 is a flowchart for describing an operation of the feature amount data generating unit 12 included in the gait measuring device 10. In the description along the flowchart of FIG. 22, the feature amount data generating unit 12 will be described as an operation subject.

In FIG. 22, first, the feature amount data generating unit 12 acquires time-series data of sensor data regarding a motion of a foot (step S101).

Next, the feature amount data generating unit 12 extracts gait waveform data for one gait cycle from the time-series data of the sensor data (step S102). The feature amount data generating unit 12 detects a heel contact and a toe off from the time-series data of the sensor data. The feature amount data generating unit 12 extracts time-series data of a section between consecutive heel contacts as gait waveform data for one gait cycle.

Next, the feature amount data generating unit 12 normalizes the extracted gait waveform data for one gait cycle (step S103). The feature amount data generating unit 12 normalizes the gait waveform data for one gait cycle to a gait cycle of 0 to 100% (first normalization). Further, the feature amount data generating unit 12 normalizes the ratio of a stance phase to a swing phase in the gait waveform data for one gait cycle having subjected to the first normalization to 60:40 (second normalization).

Next, the feature amount data generating unit 12 extracts a feature amount from the gait phase used for estimating the mobility with respect to the normalized gait waveform (step S104). For example, the feature amount data generating unit 12 extracts a feature amount to be input to an estimation model constructed in advance.

Next, the feature amount data generating unit 12 generates feature amounts for each gait phase cluster using the extracted feature amount (step S105).

Next, the feature amount data generating unit 12 integrates the feature amounts for each gait phase cluster to generate feature amount data for one gait cycle (step S106).

Next, the feature amount data generating unit 12 outputs the generated feature amount data to the mobility estimation device 13 (step S107).

### [Mobility Estimation Device]

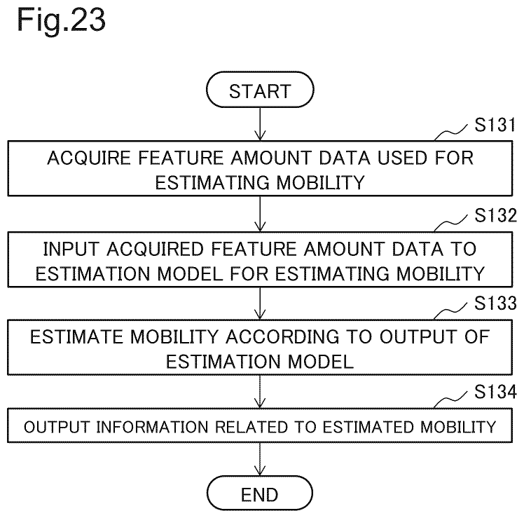

FIG. 23 is a flowchart for describing the operation of the mobility estimation device 13. In the description along the flowchart of FIG. 23, the mobility estimation device 13 will be described as an operation subject.

In FIG. 23, first, the mobility estimation device 13 acquires feature amount data generated using sensor data regarding the movement of the foot (step S131).

Next, the mobility estimation device 13 inputs the acquired feature amount data to an estimation model for estimating the mobility (TUG required time) (step S132).

Next, the mobility estimation device 13 estimates the mobility of the user depending on the output (estimated value) from the estimation model (step S133). For example, the mobility estimation device 13 estimates the TUG required time of the user as the mobility.

Next, the mobility estimation device 13 outputs information related to the estimated mobility (step S134). For example, the mobility is output to a terminal device (not illustrated) carried by the user. For example, the mobility is output to a system that executes processing using the mobility.

### (Application Example)

Next, an application example according to the present example embodiment will be described with reference to the drawings. In the following application example, an example in which the function of the mobility estimation device 13 installed in the mobile terminal carried by the user estimates the mobility using the feature amount data measured by the gait measuring device 10 arranged in the shoe will be described.

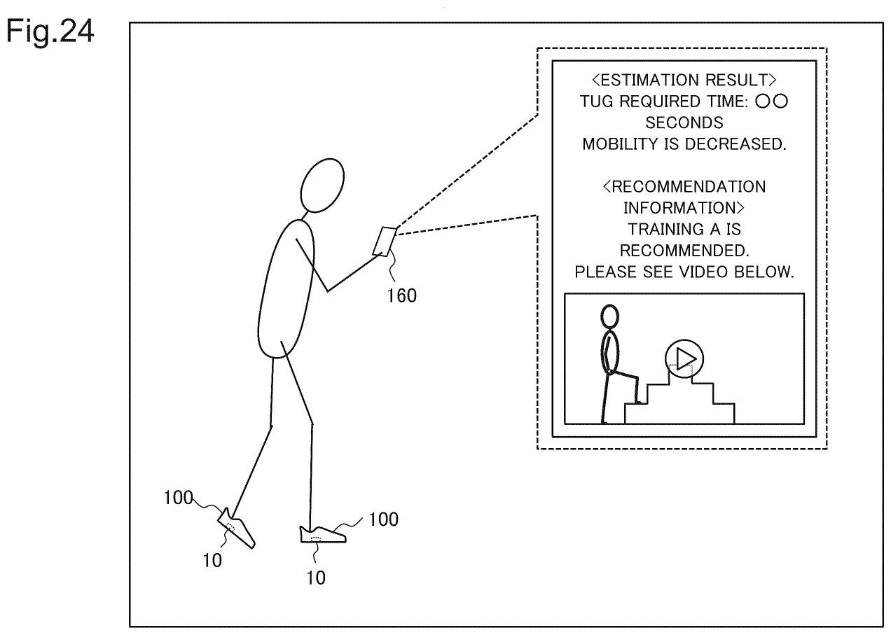

FIG. 24 is a conceptual diagram illustrating an example in which an estimation result by the mobility estimation device 13 is displayed on the screen of a mobile terminal 160 carried by the user walking while wearing the shoes 100 on which the gait measuring device 10 is arranged. FIG. 24 is an example in which information corresponding to an estimation result of mobility using the feature amount data corresponding to sensor data measured while the user is walking is displayed on the screen of the mobile terminal 160.

FIG. 24 illustrates an example in which information corresponding to the estimated value of the required TUG required time, which is the mobility, is displayed on the screen of the mobile terminal 160. In the example of FIG. 24, the estimated value of the TUG required time is displayed on a display unit of the mobile terminal 160 as the estimation result of the mobility. In the example of FIG. 24, information regarding the estimation result of the mobility of “Mobility is decreased.” is displayed on the display unit of the mobile terminal 160 in accordance with the estimated value of the TUG required time, which is the mobility. In the example of FIG. 24, recommendation information based on an estimation result of the mobility of “Training A is recommended. Please see the video below.” is displayed on the display unit of the mobile terminal 160 in accordance with the estimated value of the TUG required time, which is the mobility. The user who has confirmed the information displayed on the display unit of the mobile terminal 160 can practice training leading to an increase in mobility by exercising with reference to the video of the training A according to the recommendation information.

As described above, the mobility estimation system of the present example embodiment includes the gait measuring device and the mobility estimation device. The gait measuring device includes a sensor and a feature amount data generating unit. The sensor includes an acceleration sensor and an angular velocity sensor. The sensor measures a spatial acceleration using the acceleration sensor. The sensor measures a spatial angular velocity using the angular velocity sensor. The sensor uses the measured spatial acceleration and spatial angular velocity to generate sensor data regarding a motion of a foot. The sensor outputs the generated sensor data to the feature amount data generating unit. The feature amount data generating unit acquires time-series data of sensor data regarding the motion of the foot. The feature amount data generating unit extracts gait waveform data for one gait cycle from the time-series data of the sensor data. The feature amount data generating unit normalizes the extracted gait waveform data. The feature amount data generating unit extracts, from the normalized gait waveform data, a feature amount used for estimating the mobility from a gait phase cluster including at least one temporally continuous gait phase. The feature amount data generating unit generates feature amount data including the extracted feature amount. The feature amount data generating unit outputs the generated feature amount data.

The mobility estimation device includes a data acquisition unit, a storage unit, an estimation unit, and an output unit. The data acquisition unit acquires feature amount data including a feature amount used for estimating the mobility of the user extracted from sensor data regarding the movement of the foot of the user. The storage unit stores an estimation model that outputs a mobility index based on an input of the feature amount data. The estimation unit inputs the acquired feature amount data to the estimation model to estimate the mobility of the user. The output unit outputs information on the estimated mobility.

The mobility estimation system of the present example embodiment estimates the mobility of the user using the feature amount extracted from the sensor data regarding the movement of the foot of the user. Thus, by the mobility estimation system of the present example embodiment, the mobility can be appropriately estimated in daily life without using an instrument for measuring the mobility.

In one aspect of the present example embodiment, the data acquisition unit acquires the feature amount data including the feature amount extracted from the gait waveform data generated using the time-series data of the sensor data regarding the movement of the foot. The data acquisition unit acquires feature amount data including a feature amount used to estimate a score value of the standing and sitting test as the mobility index. According to the present aspect, by using the sensor data regarding the movement of the foot, the mobility can be appropriately estimated in daily life without using an instrument for measuring the mobility.

In one aspect of the present example embodiment, the storage unit stores an estimation model generated by machine learning using teacher data related to a plurality of subjects. The estimation model is generated by machine learning using teacher data having a feature amount used for estimating the mobility index as an explanatory variable and the mobility indexes of a plurality of subjects as an objective variable. The estimation unit inputs the feature amount data acquired regarding the user to the estimation model. The estimation unit estimates the mobility of the user in accordance with the mobility index of the user output from the estimation model. According to the present aspect, it is possible to appropriately estimate the mobility in daily life without using an instrument for measuring the mobility.

In one aspect of the present example embodiment, the storage unit stores the estimation model machine-learned using the explanatory variables including the attribute data (age) of the subject. The estimation unit inputs the feature amount data and the attribute data (age) regarding the user to the estimation model. The estimation unit estimates the mobility of the user in accordance with the mobility index of the user output from the estimation model. In the present aspect, the mobility is estimated including attribute data (age) that affects the mobility. Thus, according to the present aspect, the mobility can be measured with higher accuracy.

In one aspect of the present example embodiment, the storage unit stores an estimation model generated by machine learning using teacher data related to a plurality of subjects. The estimation model is a model generated by machine learning using teacher data having a feature amount extracted from the gait waveform data of the plurality of subjects as an explanatory variable and a mobility index of the plurality of subjects as an objective variable. For example, a feature amount regarding the activity of the gluteus medius muscle extracted from the mid-stance period is included in the explanatory variables. For example, a feature amount regarding the quadriceps femoris extracted from a section from the pre-swing period to the initial swing period is included in the explanatory variables. For example, the feature amount regarding the activity of the tibialis anterior muscle extracted from the mid-swing period is included in the explanatory variables. The estimation unit inputs feature amount data acquired in accordance with a gait of the user to the estimation model. The estimation unit estimates the mobility of the user in accordance with the mobility index of the user output from the estimation model. According to the present aspect, the mobility more suitable for the physical activity can be estimated by using the estimation model in which the feature amount based on the muscle activity that affects the mobility is machine-learned.

In one aspect of the present example embodiment, the storage unit stores, for a plurality of subjects, an estimation model generated by machine learning using teacher data having a plurality of feature amounts extracted from gait waveform data as explanatory variables and a mobility regarding a mobility index of the subject as an objective variable. For example, a feature amount extracted from the initial swing period of the gait waveform data of the lateral acceleration is included in the explanatory variables. For example, the feature amount extracted from the pre-swing period of the gait waveform data of the angular velocity in the sagittal plane is included in the explanatory variables. For example, the feature amount extracted from an early stage of the mid-stance period and the early stage of the mid-swing period of the gait waveform data of the angular velocity in the horizontal plane is included in the explanatory variable. For example, the feature amount extracted from the mid-swing period of the gait waveform data of the angle in the coronal plane is included in the explanatory variable.

The data acquisition unit acquires feature amount data including a feature amount extracted in accordance with a gait of the user. For example, the data acquisition unit acquires a feature amount of the initial swing period of the gait waveform data of the lateral acceleration. For example, the data acquisition unit acquires the feature amount of the pre-swing period in the gait waveform data of the angular velocity in the sagittal plane. For example, the data acquisition unit acquires feature amounts of an early stage of the mid-stance period and the early stage of the mid-swing period of the gait waveform data of the angular velocity in the horizontal plane. For example, the data acquisition unit acquires feature amounts of an early stage of the mid-stance period and the early stage of the mid-swing period of the gait waveform data of the angular velocity in the horizontal plane. The estimation unit inputs the acquired feature amount data to the estimation model. The estimation unit estimates the mobility of the user in accordance with the mobility index of the user output from the estimation model. According to the present aspect, by using the estimation model in which the feature amount extracted from the gait waveform data including the feature based on the activity of the muscle that affects the mobility is machine-learned, the mobility more suitable for the physical activity can be estimated using the sensor data regarding the movement of the foot.

In an aspect of the present example embodiment, the mobility estimation device is implemented in a terminal device having a screen visually recognizable by a user. For example, the mobility estimation device displays information regarding the mobility estimated in accordance with the movement of the foot of the user on the screen of the terminal device. For example, the mobility estimation device displays recommendation information based on the mobility estimated in accordance with the movement of the foot of the user on the screen of the terminal device. For example, the mobility estimation device displays a video related to training for training a body part related to mobility on a screen of the terminal device as recommendation information based on the mobility estimated in accordance with the movement of the foot of the user. According to the present aspect, by displaying the mobility estimated according to the feature amount extracted from the sensor data regarding the movement of the foot of the user on the screen visually recognizable by the user, the user can confirm the information according to the mobility of the user.

### Second Example Embodiment

Next, a machine learning system according to a second example embodiment will be described with reference to the drawings. The machine learning system according to the present example embodiment generates an estimation model for estimating the mobility according to the input of the feature amount by machine learning using the feature amount data extracted from the sensor data measured by the gait measuring device.

### (Configuration)

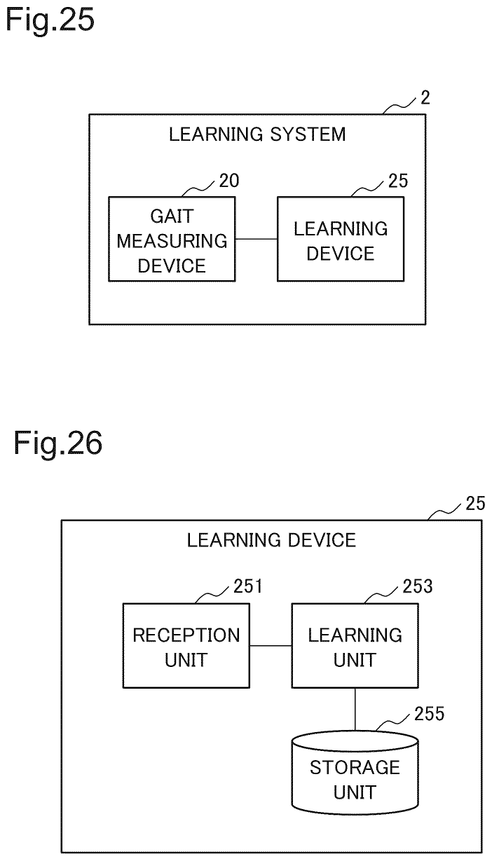

FIG. 25 is a block diagram illustrating an example of a configuration of the machine learning system 2 according to the present example embodiment. The machine learning system 2 includes a gait measuring device 20 and a machine learning device 25. The gait measuring device 20 and the machine learning device 25 may be connected by wire or wirelessly. The gait measuring device 20 and the machine learning device 25 may be configured by a single device. The machine learning system 2 may be configured only by the machine learning device 25 except for the gait measuring device 20 from the configuration of the machine learning system 2. Although only one gait measuring device 20 is illustrated in FIG. 25, one (two in total) gait measuring device 20 may be arranged on each of the left and right feet. The machine learning device 25 may be configured not to be connected to the gait measuring device 20 but to execute machine learning using the feature amount data generated in advance by the gait measuring device 20 and stored in the database.

The gait measuring device 20 is installed on at least one of the left and right legs. The gait measuring device 20 has a configuration similar to that of the gait measuring device 10 of the first example embodiment. The gait measuring device 20 includes an acceleration sensor and an angular velocity sensor. The gait measuring device 20 converts the measured physical quantity into digital data (also referred to as sensor data). The gait measuring device 20 generates normalized gait waveform data for one gait cycle from the time-series data of the sensor data. The gait measuring device 20 generates feature amount data used for estimating a mobility to be estimated. The gait measuring device 20 transmits the generated feature amount data to the machine learning device 25. The gait measuring device 20 may be configured to transmit the feature amount data to a database (not illustrated) accessed by the machine learning device 25. The feature amount data accumulated in the database is used for machine learning by the machine learning device 25.

The machine learning device 25 receives the feature amount data from the gait measuring device 20. When using the feature amount data accumulated in the database (not illustrated), the machine learning device receives the feature amount data from the database. The machine learning device 25 executes machine learning using the received feature amount data. For example, the machine learning device 25 learns teacher data in which feature amount data extracted from a plurality of pieces of subject gait waveform data is set as an explanatory variable and a value related to mobility according to the feature amount data is set as an objective variable. The machine learning algorithm executed by the machine learning device 25 is not particularly limited. The machine learning device 25 generates an estimation model learned using teacher data related to a plurality of subjects. The machine learning device 25 stores the generated estimation model. The estimation model learned by the machine learning device 25 may be stored in a storage device outside the machine learning device 25.

### [Machine Learning Device]

Next, details of the machine learning device 25 will be described with reference to the drawings. FIG. 26 is a block diagram illustrating an example of a detailed configuration of the machine learning device 25. The machine learning device 25 includes a reception unit 251, a machine learning unit 253, and a storage unit 255.

The reception unit 251 receives the feature amount data from the gait measuring device 20. The reception unit 251 outputs the received feature amount data to the machine learning unit 253. The reception unit 251 may receive the feature amount data from the gait measuring device via a wire such as a cable, or may receive the feature amount data from the gait measuring device 20 via wireless communication. For example, the reception unit 251 is configured to receive the feature amount data from the gait measuring device 20 via a wireless communication function (not illustrated) conforming to a standard such as Bluetooth (registered trademark) or WiFi (registered trademark). The communication function of the reception unit 251 may conform to a standard other than Bluetooth (registered trademark) or WiFi (registered trademark).

The machine learning unit 253 acquires the feature amount data from the reception unit 251. The machine learning unit 253 executes machine learning using the acquired feature amount data. For example, the machine learning unit 253 learns a data set in which the feature amount data extracted from the sensor data measured according to the movement of the foot of the subject is set as an explanatory variable and the TUG required time of the subject is set as an objective variable as teacher data. For example, the machine learning unit 253 generates an estimation model that estimates the TUG required time according to the input of the feature amount data learned for a plurality of subjects. For example, the machine learning unit 253 generates an estimation model according to attribute data (age). For example, the machine learning unit 253 generates an estimation model for estimating the TUG required time as the mobility using the feature amount data extracted from the sensor data measured according to the movement of the foot of the subject and the attribute data (age) of the subject as explanatory variables. The machine learning unit 253 stores estimation models learned for a plurality of subjects in the storage unit 255.

For example, the machine learning unit 253 executes machine learning using a linear regression algorithm. For example, the machine learning unit 253 executes machine learning using an algorithm of a support vector machine (SVM). For example, the machine learning unit 253 executes machine learning using a Gaussian process regression (GPR) algorithm. For example, the machine learning unit 253 executes machine learning using a random forest (RF) algorithm. For example, the machine learning unit 253 may execute unsupervised machine learning of classifying a subject who is a generation source of the feature amount data according to the feature amount data. The machine learning algorithm executed by the machine learning unit 253 is not particularly limited.

The machine learning unit 253 may execute machine learning using the gait waveform data for one gait cycle as an explanatory variable. For example, the machine learning unit 253 executes supervised machine learning in which the acceleration in the three-axis direction, the angular velocity around the three axes, and the gait waveform data of the angle (posture angle) around the three axes are set as explanatory variables and the correct value of the mobility that is the estimation target is set as an objective variable. For example, in a case where the gait phase is set in increments of 1% in a 0 to 100% gait cycle, the machine learning unit 253 learns by using 909 explanatory variables.

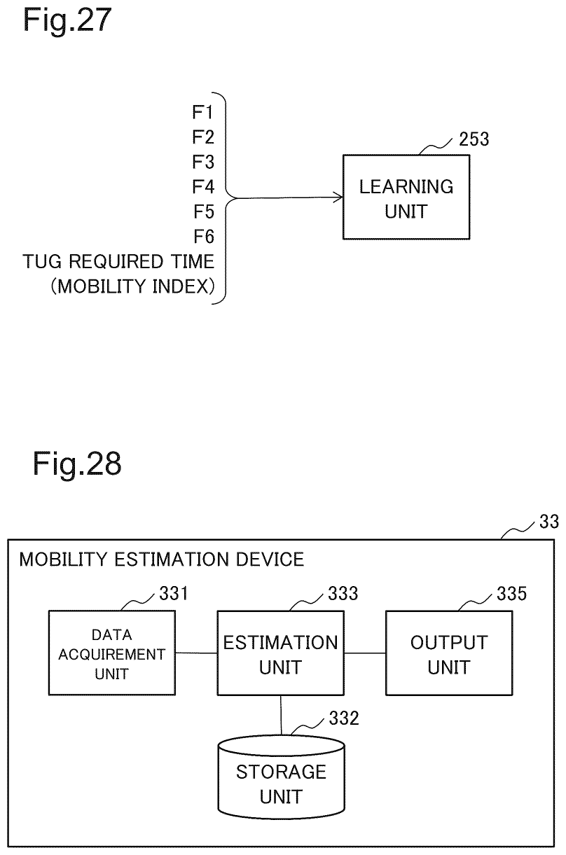

FIG. 27 is a conceptual diagram for describing machine learning for generating an estimation model. FIG. 27 is a conceptual diagram illustrating an example of causing the machine learning unit 253 to learn a data set of the feature amounts F1 to F6 which are explanatory variables and the TUG required time (mobility index) which is an objective variable as teacher data. For example, the machine learning unit 253 learns data related to a plurality of subjects, and generates an estimation model that outputs an output (estimated value) related to a TUG required time (mobility index) according to an input of a feature amount extracted from sensor data.

The storage unit 255 stores estimation models machine-learned for a plurality of subjects. For example, the storage unit 255 stores an estimation model for estimating the mobility machine-learned for a plurality of subjects. For example, the estimation model stored in the storage unit 255 is used for estimating the mobility by the mobility estimation device 13 of the first example embodiment.

As described above, the machine learning system of the present example embodiment includes the gait measuring device and the machine learning device. The gait measuring device acquires time-series data of sensor data regarding a motion of a foot. The gait measuring device extracts gait waveform data for one gait cycle from the time-series data of the sensor data, and normalizes the extracted gait waveform data. The gait measuring device extracts a feature amount used for estimating the mobility of the user from the normalized gait waveform data from a gait phase cluster configured by at least one temporally continuous gait phase. The gait measuring device generates feature amount data including the extracted feature amount. The gait measuring device outputs the generated feature amount data to the machine learning device.

The machine learning device includes a reception unit, a machine learning unit, and a storage unit. The reception unit acquires the feature amount data generated by the gait measuring device. The machine learning unit executes machine learning using the feature amount data. The machine learning unit generates the estimation model that outputs the mobility in accordance with the input of the feature amount (second feature amount) of the gait phase cluster extracted from the time-series data of the sensor data measured along with the gait of the user. The estimation model generated by the machine learning unit is stored in the storage unit.

The machine learning system of the present example embodiment generates an estimation model by using the feature amount data measured by the gait measuring device. Thus, according to the present aspect, it is possible to generate an estimation model capable of appropriately estimating the mobility in daily life without using an instrument for measuring the mobility.

### Third Example Embodiment

Next, a mobility estimation device according to a third example embodiment will be described with reference to the drawings. The mobility estimation device of the present example embodiment has a simplified configuration of the mobility estimation device included in the mobility estimation system of the first example embodiment.

FIG. 28 is a block diagram illustrating an example of a configuration of the mobility estimation device 33 according to the present example embodiment. The mobility estimation device 33 includes a data acquisition unit 331, a storage unit 332, an estimation unit 333, and an output unit 335.

The data acquisition unit 331 acquires feature amount data including a feature amount used for estimating a mobility index of the user, the feature amount data being extracted from sensor data regarding the movement of the foot of the user. The storage unit 332 stores an estimation model that outputs a mobility index based on the input of the feature amount data. The estimation unit 333 inputs the acquired feature amount data to the estimation model, and estimates the mobility of the user in accordance with the mobility index output from the estimation model. The output unit 335 outputs information on the estimated mobility.

As described above, in the present example embodiment, the mobility of the user is estimated using the feature amount extracted from the sensor data regarding the movement of the foot of the user. Thus, according to the present example embodiment, it is possible to appropriately estimate the mobility in daily life without using an instrument for measuring the mobility.

### (Hardware)

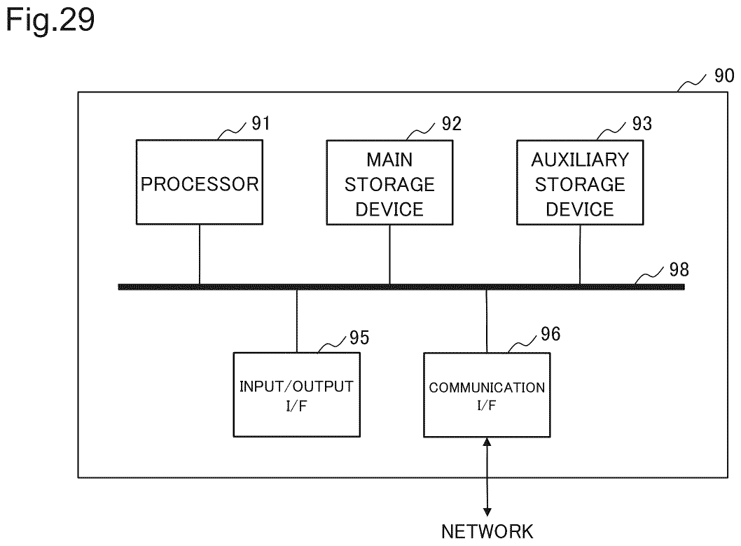

Here, a hardware configuration for executing control and processing according to each example embodiment of the present disclosure will be described using the information processing device 90 of FIG. 29 as an example. The information processing device 90 in FIG. 29 is a configuration example for executing control and processing of each example embodiment, and does not limit the scope of the present disclosure.

As illustrated in FIG. 29, the information processing device 90 includes a processor 91, a main storage device 92, an auxiliary storage device 93, an input-output interface 95, and a communication interface 96. In FIG. 29, the interface is abbreviated as an interface (I/F). The processor 91, the main storage device 92, the auxiliary storage device 93, the input-output interface 95, and the communication interface 96 are data-communicably connected to each other via a bus 98. The processor 91, the main storage device 92, the auxiliary storage device 93, and the input-output interface 95 are connected to a network such as the Internet or an intranet via the communication interface 96.

The processor 91 develops the program stored in the auxiliary storage device 93 or the like in the main storage device 92. The processor 91 executes the program developed in the main storage device 92. In the present example embodiment, it is only required to use a software program installed in the information processing device 90. The processor 91 executes control and processing according to each example embodiment.

The main storage device 92 has an area in which a program is developed. A program stored in the auxiliary storage device 93 or the like is developed in the main storage device 92 by the processor 91. The main storage device 92 is implemented by, for example, a volatile memory such as a dynamic random access memory (DRAM). A nonvolatile memory such as a magnetoresistive random access memory (MRAM) may be configured and added as the main storage device 92.

The auxiliary storage device 93 stores various data such as programs. The auxiliary storage device 93 is implemented by a local disk such as a hard disk or a flash memory. In addition, the main storage device 92 may be configured to store various data, and the auxiliary storage device 93 may be omitted.

The input-output interface 95 is an interface for connecting the information processing device 90 and a peripheral device based on a standard or a specification. The communication interface 96 is an interface for connecting to an external system or device through a network such as the Internet or an intranet based on a standard or a specification. The input-output interface 95 and the communication interface 96 may be shared as an interface connected to an external device.

Input devices such as a keyboard, a mouse, and a touch panel may be connected to the information processing device 90 as necessary. These input devices are used to input information and settings. In a case where the touch panel is used as the input device, the display screen of the display device may also serve as the interface of the input device. Data communication between the processor 91 and the input device is only required to be mediated by the input-output interface 95.

The information processing device 90 may be provided with a display device for displaying information. In a case where a display device is provided, the information processing device 90 preferably includes a display control device (not illustrated) for controlling display of the display device. The display device is only required to be connected to the information processing device 90 via the input-output interface 95.

The information processing device 90 may be provided with a drive device. The drive device mediates reading of data and a program from a recording medium, writing of a processing result of the information processing device 90 to the recording medium, and the like between the processor 91 and the recording medium (program recording medium). The drive device only needs to be connected to the information processing device 90 via the input-output interface 95.

The above is an example of a hardware configuration for enabling control and processing according to each example embodiment of the present invention. The hardware configuration of FIG. 29 is an example of a hardware configuration for executing control and processing according to each example embodiment, and does not limit the scope of the present invention. A program for causing a computer to execute control and processing according to each example embodiment is also included in the scope of the present invention. Further, a program storage medium in which the program according to each example embodiment is stored is also included in the scope of the present invention. The storage medium can be achieved by, for example, an optical storage medium such as a compact disc (CD) or a digital versatile disc (DVD). The recording medium may be implemented by a semiconductor recording medium such as a universal serial bus (USB) memory or a secure digital (SD) card. The recording medium may be implemented by a magnetic recording medium such as a flexible disk, or another recording medium. When a program executed by the processor is recorded in a recording medium, the recording medium corresponds to a program recording medium.

The components of each example embodiment may be combined in any manner. The components of each example embodiment may be implemented by software or may be implemented by a circuit.

While the present invention has been particularly illustrated and described with reference to example embodiments thereof, the invention is not limited to these example embodiments. It will be understood by those of ordinary skill in the art that various changes in form and details may be made therein without departing from the spirit and scope of the present invention as defined by the claims.

Some or all of the example embodiments described above may also be described as in the following supplementary notes, but are not limited to the following.

### (Supplementary Note 1)

A mobility estimation device including:
a data acquisition unit that acquires feature amount data including a feature amount used for estimating a mobility of a user, the feature amount data being extracted from sensor data regarding a movement of a foot of the user;
a storage unit that stores an estimation model that outputs a mobility index based on an input of the feature amount data;
an estimation unit that inputs the acquired feature amount data to the estimation model and estimates the mobility of the user in accordance with the mobility index output from the estimation model; and
an output unit that outputs information regarding the estimated mobility of the user.

### (Supplementary Note 2)

The mobility estimation device according to supplementary note 1, in which
the data acquisition unit
acquires the feature amount data including a feature amount used to estimate a grade value of a time up and go (TUG) test as the mobility index, the feature amount data being extracted from gait waveform data generated using time-series data of the sensor data regarding a movement of a foot.

### (Supplementary Note 3)

The mobility estimation device according to supplementary note 2, in which
the storage unit
stores, regarding a plurality of subjects, the estimation model generated by machine learning using teacher data in which a feature amount used to estimate the mobility index is set as an explanatory variable and the mobility index for the plurality of subjects is set as an objective variable, and
the estimation unit
inputs the feature amount data acquired regarding the user to the estimation model, and estimates the mobility of the user in accordance with the mobility index of the user output from the estimation model.

### (Supplementary Note 4)

The mobility estimation device according to supplementary note 3, in which
the storage unit
stores the estimation model machine-learned using explanatory variables including ages of the plurality of subjects, and
the estimation unit
inputs the feature amount data and an age related to the user are input to the estimation model, and estimates the mobility of the user in accordance with the mobility index of the user output from the estimation model.

### (Supplementary Note 5)

The mobility estimation device according to supplementary note 3 or 4, in which
the storage unit
stores the estimation model generated by machine learning using teacher data in which, with respect to the gait waveform data of the plurality of subjects, a feature amount regarding an activity of the gluteus medius muscle extracted from a mid-stance period, a feature amount regarding a quadriceps femoris extracted from a section from a pre-swing period to an initial swing period, and a feature amount regarding an activity of a tibialis anterior muscle extracted from a mid-swing period are set as explanatory variables, and the mobility indexes of the plurality of subjects are set as objective variables, and
the estimation unit
inputs the feature amount data acquired in accordance with a gait of the user to the estimation model, and estimates the mobility of the user in accordance with the mobility index of the user output from the estimation model.

### (Supplementary Note 6)

The mobility estimation device according to supplementary note 5, in which
the storage unit
stores the estimation model generated by machine learning using teacher data in which, with respect to the plurality of subjects, a feature amount extracted from an initial swing period of the gait waveform data of a lateral acceleration, a feature amount extracted from a pre-swing period of the gait waveform data of an angular velocity in a sagittal plane, a feature amount extracted from a mid-stance period of the gait waveform data of an angular velocity in a coronal plane, a feature amount extracted from an early stage of a mid-stance period and an early stage of a mid-swing period of the gait waveform data of an angle in a horizontal plane, and a feature amount extracted from a mid-swing period of the gait waveform data of an angle in the coronal plane are set as explanatory variables, and the mobility indexes of the plurality of subjects as objective variables,
the data acquisition unit
acquires the feature amount data including a feature amount at an initial swing period of the gait waveform data of a lateral acceleration, a feature amount at a pre-swing period of the gait waveform data of an angular velocity in a sagittal plane, a feature amount at a mid-stance period of the gait waveform data of an angular velocity in a coronal plane, a feature amount at an early stage of a mid-stance period and an early stage of a mid-swing period of the gait waveform data of an angle in a horizontal plane, and a feature amount at a mid-swing period of the gait waveform data of an angle in the coronal plane extracted in accordance with a gait of the user, and
the estimation unit
inputs the acquired feature amount data to the estimation model, and estimates the mobility of the user in accordance with the mobility index of the user output from the estimation model.

### (Supplementary Note 7)

The mobility estimation device according to any one of supplementary notes 3 to 6, in which
the estimation unit
estimates information regarding the mobility of the user in accordance with the mobility index estimated for the user, and the output unit
outputs information regarding the estimated mobility.

### (Supplementary Note 8)

A mobility estimation system including:
the mobility estimation device according to any one of supplementary notes 1 to 7; and
a gait measuring device including a sensor that is installed on footwear of a user who is an estimation target of mobility, and measures a spatial acceleration and a spatial angular velocity, generates sensor data regarding a movement of a foot using the spatial acceleration and the spatial angular velocity that have been measured, and outputs the generated sensor data, and a feature amount data generating unit that acquires time-series data of the sensor data including a feature of a gait, extract gait waveform data for one gait cycle from the time-series data of the sensor data, normalizes the extracted gait waveform data, extracts a feature amount used for estimating the mobility from a gait phase cluster including at least one temporally continuous gait phase from the normalized gait waveform data, generates feature amount data including the extracted feature amount, and outputs the generated feature amount data to the mobility estimation device.

### (Supplementary Note 9)

The mobility estimation system according to supplementary note 8, in which
the mobility estimation device
is mounted in a terminal device having a screen visible by the user, and
causes information regarding the mobility estimated in accordance with a movement of a foot of the user to be displayed on a screen of the terminal device.

### (Supplementary Note 10)

The mobility estimation system according to supplementary note 9, in which
the mobility estimation device
causes recommendation information based on the mobility estimated in accordance with the movement of the foot of the user to be displayed on a screen of the terminal device.

### (Supplementary Note 11)

The mobility estimation system according to supplementary note 10, in which
the mobility estimation device
causes a moving image related to training for training a body part related to the mobility to be displayed on a screen of the terminal device as the recommendation information based on the mobility estimated in accordance with the movement of the foot of the user.

### (Supplementary Note 12)

A mobility estimation method including, by a computer:
acquiring feature amount data including a feature amount used for estimating a mobility of a user, the feature amount data being extracted from sensor data regarding a movement of a foot of the user;
inputting the acquired feature amount data to an estimation model that outputs a mobility index based on an input of the feature amount data;
estimating the mobility of the user in accordance with the mobility index output from the estimation model; and
outputting information regarding the estimated mobility of the user.

### (Supplementary Note 13)

A non-transitory recording medium recording a program for causing a computer to execute:
processing of acquiring feature amount data including a feature amount used for estimating a mobility of a user, the feature amount data being extracted from sensor data regarding a movement of a foot of the user;
processing of inputting the acquired feature amount data to an estimation model that outputs a mobility index based on an input of the feature amount data;
processing of estimating the mobility of the user in accordance with the mobility index output from the estimation model; and
processing of outputting information regarding the estimated mobility of the user.

### REFERENCE SIGNS LIST

1 mobility estimation system
2 machine learning system
10, 20 gait measuring device
11 sensor
12 feature amount data generating unit
13 mobility estimation device
25 machine learning device
111 acceleration sensor
112 angular velocity sensor
121 acquisition unit
122 normalization unit
123 extraction unit
125 generation unit
127 feature amount data output unit
131, 331 data acquisition unit
132, 332 storage unit
133, 333 estimation unit
135, 335 output unit
251 reception unit
253 machine learning unit
255 storage unit

What is claimed is:

1. A mobility estimation device comprising:
a storage configured to store an estimation model that outputs a mobility index corresponding to input of feature amount data used for estimating a mobility;
a memory storing instructions; and
a processor connected to the memory and configured to execute the instructions to:
acquire feature amount data including a feature amount used for estimating a mobility of a user, the feature amount data being extracted from sensor data regarding a movement of a foot of the user;
input the acquired feature amount data to the estimation model and estimate the mobility of the user in accordance with the mobility index output from the estimation model; and
output information regarding the estimated mobility of the user.

2. The mobility estimation device according to claim 1, wherein
the processor is configured to execute the instructions to
acquire the feature amount data including a feature amount used to estimate a grade value of a time up and go (TUG) test as the mobility index, the feature amount data being extracted from gait waveform data generated using time-series data of the sensor data regarding a movement of a foot.

3. The mobility estimation device according to claim 2, wherein
the storage stores, regarding a plurality of subjects, the estimation model generated by machine learning using teacher data in which a feature amount used to estimate the mobility index is set as an explanatory variable and the mobility index for the plurality of subjects is set as an objective variable, and
the processor is configured to execute the instructions to
input the feature amount data acquired regarding the user to the estimation model, and
estimate the mobility of the user in accordance with the mobility index of the user output from the estimation model.

4. The mobility estimation device according to claim 3, wherein
the storage means stores the estimation model machine-learned using explanatory variables including ages of the plurality of subjects, and
the processor is configured to execute the instructions to
input the feature amount data and an age related to the user to the estimation model, and
estimate the mobility of the user in accordance with the mobility index of the user output from the estimation model.

5. The mobility estimation device according to claim 3, wherein
the storage stores the estimation model generated by machine learning using teacher data in which, with respect to the gait waveform data of the plurality of subjects, a feature amount regarding an activity of the gluteus medius muscle extracted from a mid-stance period, a feature amount regarding a quadriceps femoris extracted from a section from a pre-swing period to an initial swing period, and a feature amount regarding an activity of a tibialis anterior muscle extracted from a mid-swing period are set as explanatory variables, and the mobility indexes of the plurality of subjects are set as objective variables, and
the processor is configured to execute the instructions to
input the feature amount data acquired in accordance with a gait of the user to the estimation model, and
estimate the mobility of the user in accordance with the mobility index of the user output from the estimation model.

6. The mobility estimation device according to claim 5, wherein
the storage stores the estimation model generated by machine learning using teacher data in which, with respect to the plurality of subjects, a feature amount extracted from an initial swing period of the gait waveform data of a lateral acceleration, a feature amount extracted from a pre-swing period of the gait waveform data of an angular velocity in a sagittal plane, a feature amount extracted from a mid-stance period of the gait waveform data of an angular velocity in a coronal plane, a feature amount extracted from an early stage of a mid-stance period and an early stage of a mid-swing period of the gait waveform data of an angle in a horizontal plane, and a feature amount extracted from a mid-swing period of the gait waveform data of an angle in the coronal plane are set as explanatory variables, and the mobility indexes of the plurality of subjects as objective variables,
the processor is configured to execute the instructions to
acquire the feature amount data including a feature amount at an initial swing period of the gait waveform data of a lateral acceleration, a feature amount at a pre-swing period of the gait waveform data of an angular velocity in a sagittal plane, a feature amount at a mid-stance period of the gait waveform data of an angular velocity in a coronal plane, a feature amount at an early stage of a mid-stance period and an early stage of a mid-swing period of the gait waveform data of an angle velocity in a horizontal plane, and a feature amount at a mid-swing period of the gait waveform data of an angle in the coronal plane extracted in accordance with a gait of the user, and
input the acquired feature amount data to the estimation model, and
estimate the mobility of the user in accordance with the mobility index of the user output from the estimation model.

7. The mobility estimation device according to claim 3, wherein
the processor is configured to execute the instructions to
estimate information regarding the mobility of the user in accordance with the mobility index estimated for the user, and
output information regarding the estimated mobility.

8. A mobility estimation system comprising:
the mobility estimation device according to claim 1; and
a gait measuring device comprising
a sensor that is installed on footwear of a user who is an estimation target of mobility, and measures a spatial acceleration and a spatial angular velocity, generates sensor data regarding a movement of a foot using the spatial acceleration and the spatial angular velocity that have been measured, and output the generated sensor data, and
a memory storing instructions; and
a processor connected to the memory and configured to execute the instructions to
acquire time-series data of the sensor data including a feature of a gait,
extract gait waveform data for one gait cycle from the time-series data of the sensor data,
normalize the extracted gait waveform data,
extract a feature amount used for estimating the mobility from a gait phase cluster including at least one temporally continuous gait phase from the normalized gait waveform data,
generate feature amount data including the extracted feature amount, and
output the generated feature amount data to the mobility estimation device.

9. The mobility estimation system according to claim 8, wherein
the mobility estimation device is mounted in a terminal device having a screen visible by the user, and
the processer of the mobility estimation device is configured to execute the instructions to cause information regarding the mobility estimated in accordance with a movement of a foot of the user to be displayed on a screen of the terminal device.

10. The mobility estimation system according to claim 9, wherein
the processer of the mobility estimation device is configured to execute the instructions to cause recommendation information based on the mobility estimated in accordance with the movement of the foot of the user to be displayed on a screen of the terminal device.

11. The mobility estimation system according to claim 10, wherein
the processer of the mobility estimation device is configured to execute the instructions to cause a moving image related to training for training a body part related to the mobility to be displayed on a screen of the terminal device as the recommendation information based on the mobility estimated in accordance with the movement of the foot of the user.

12. A mobility estimation method comprising, by a computer:
acquiring feature amount data including a feature amount used for estimating a mobility of a user, the feature amount data being extracted from sensor data regarding a movement of a foot of the user;
inputting the acquired feature amount data to an estimation model that outputs a mobility index based on an input of the feature amount data;
estimating the mobility of the user in accordance with the mobility index output from the estimation model; and
outputting information regarding the estimated mobility of the user.

13. A non-transitory recording medium recording a program for causing a computer to execute:
processing of acquiring feature amount data including a feature amount used for estimating a mobility of a user, the feature amount data being extracted from sensor data regarding a movement of a foot of the user;
processing of inputting the acquired feature amount data to an estimation model that outputs a mobility index based on an input of the feature amount data;
processing of estimating the mobility of the user in accordance with the mobility index output from the estimation model; and
processing of outputting information regarding the estimated mobility of the user.

14. The mobility estimation system according to claim 10, wherein
the processor of the mobility estimation device is configured to execute the instructions to
cause the recommendation information that supports the user for making decision about taking an action to be displayed on the screen of the terminal device.
